(19) United States
(12) Patent Application Publication
(10) Pub. No.: US 2026/0060572 A1
(43) Pub. Date: Mar. 5, 2026

(54) METHODS, SYSTEMS, AND COMPUTER READABLE MEDIA FOR DETECTING AND MONITORING GAIT USING A PORTABLE GAIT BIOFEEDBACK SYSTEM

(71) Applicant: The University of North Carolina at Chapel Hill, Chapel Hill, NC (US)

(72) Inventors: Jason Roy Franz, Durham, NC (US); Brian Pietrosimone, Durham, NC (US)

(21) Appl. No.: 19/104,617

(22) PCT Filed: Aug. 23, 2023
(86) PCT No.: PCT/US2023/030931

§ 371 Date: Feb. 18, 2025

Related U.S. Application Data
(60) Provisional application No. 63/400,163, filed on Aug. 23, 2022.

(51) Int. Cl. A61B 5/11; A61B 5/00

(52) U.S. Cl. CPC A61B 5/112; A61B 5/486; A61B 5/6829; A61B 5/7405; A61B 2562/0219

(57) ABSTRACT

A method for detecting and monitoring gait using a portabie gait biofeedback system includes receiving, by a central host computing device and from a plurality of wearable sensor devices, sensor signal data representative of biometric gait data of a subject. The method further includes combining, by the central host computing device, the sensor signal data from each of the wearable sensor devices to generate a gait data collection in real-time. The method further includes providing, by the central host computing device, the gait data collection to a cloud-based biometric prediction engine. The method further includes processing, by the biofeedback prediction engine, the gait data collection to generate vertical ground reaction force (vGRF) biofeedback prediction data associated with the subject in real-time.

### CROSS REFERENCE TO RELATED APPLICATIONS

This application claims the benefit of and priority to U.S. Provisional Patent Application Ser. No. 63/400,163 entitled “METHODS, SYSTEMS, AND COMPUTER READABLE MEDIA FOR DETECTING AND MONITORING GAIT USING A PORTABLE GAIT BIOFEEDBACK SYSTEM,” filed Aug. 23, 2022, the disclosure of which is incorporated herein by reference in its entirety.

### GOVERNMENT INTEREST

This invention was made with government support under Grant Number AR074094 awarded by the National Institutes of Health. The government has certain rights in the invention.

### TECHNICAL FIELD

The subject matter described herein relates to rehabilitative systems that obtain and utilize gait biofeedback measurements. More particularly, the subject matter described herein pertains to methods, systems, and computer readable media for detecting, monitoring and treating gait using a portable gait biofeedback system.

### BACKGROUND

Abnormal walking gait biomechanics are associated with the development and progression of many chronic lower extremity musculoskeletal conditions. Many of these types of musculoskeletal conditions (e.g., osteoarthritis) greatly contribute toward leading causes of global disability as well as subjecting the healthcare system to a considerable financial burden. Individuals who sustain a lower extremity joint injury (e.g., injuries to hips, knees, and/or ankles) are at high risk for developing osteoarthritis. As such, considerable personal effort and financial investment is directed to surgical and rehabilitative interventions to prevent osteoarthritis following joint injury. Unfortunately, those interventions are not specifically designed to optimize the loading of lower extremity joints, which is critical for maintaining healthy joint tissues. Notably, proper gait biomechanics act to distribute and balance forces across lower extremity joint surfaces that significantly contribute toward the preservation of joint function. Joint injury and surgery are typically accompanied by pain, swelling, and muscle weakness which can cause individuals to adopt aberrant walking biomechanics and atypical patterns of force distribution across the joint surfaces. Although these gait adaptions may allow individuals to maintain function following injury and surgery, joint tissues rapidly break down in response to atypical and unbalanced joint forces. Therefore, it is critical to promptly reestablish and maintain normal walking gait biomechanics, and thus joint tissue loading, in order to prevent the development of lower extremity osteoarthritis following joint injury.

Standard joint injury or post-surgical rehabilitation, which focuses on implementing therapeutic exercise to enhance muscle strength, is important for improving patient-reported outcomes and physical function in patients with knee injuries and osteoarthritis. However, therapeutic exercise alone does not restore normal walking biomechanics and force distribution across the joint. Unfortunately, standard rehabilitation regimens largely operate under the premise that strengthening lower extremity muscles will result in normalized joint biomechanics. Recent evidence demonstrates that patients with lower extremity joint injuries and surgeries continue to exhibit aberrant gait biomechanics after undergoing standard rehabilitation to improve muscle strength. The average person takes thousands of steps per day. As such, even subtle alterations in walking biomechanics can lead to significant deleterious changes to joint tissues over the course of months and/or years.

Current research demonstrates that clinically meaningful differences between normal and harmful gait biomechanics are relatively small and cannot be detected upon clinical examination. While physical therapists can retrain grossly abnormal walking biomechanics in the clinic, these clinicians lack precise, accessible, and evidence-based technology to detect and modify more subtle changes in walking gait biomechanics that ultimately go untreated in most patients. The detection and modification of more subtle (but equally important) alterations in walking biomechanics typically requires expensive, highly technical, and relatively inaccessible laboratory-grade motion analysis equipment. Moreover, the use of such motion analysis equipment is extremely time consuming when used by physicians to treat individual patients. Personalized prescription of joint loading cannot be feasibly implemented in the clinical management of patients with osteoarthritis because of the expensive and often immobile force-sensing equipment required. However, not detecting and treating these aberrant gait biomechanics in patients with lower extremity joint injuries can lead to considerable health consequences for patients and financial consequences for clinics that are unable to bill for specialized gait rehabilitation. Therefore, a more cost-efficient system that can easily be applied by clinicians in conventional outpatient physical therapy clinics is needed to overcome this immediate public health problem and properly treat abnormal gait biomechanics following joint injury.

With increasing clinical interest in smart gait rehabilitation therapies, instruments that enable holistic monitoring of limb kinetics outside of a laboratory setting are of growing importance. Parameters of significance for gait monitoring include inertial signals, knee moments, and the vertical ground reaction force (vGRF) of a patient's step, all of which are conventionally quantified by non-portable 3-D motion capture and force plate systems. Notably, such instruments necessitate use within a controlled lab environment with subjects walking on a split-belt treadmill. These requirements prohibit widespread clinical use and eliminate the potential for monitoring in real-world settings.

There is a critical need for inexpensive and portable systems that can monitor and prescribe evidence-based changes to critical variable of limb-level loading for the purpose of mitigating osteoarthritis onset and progression. Accordingly, there exists a need for improved methods and systems for detecting and monitoring gait using a portable gait biofeedback system.

### SUMMARY

Methods, systems, and computer readable media for detecting and monitoring gait using a portable gait biofeedback system are disclosed. A method includes receiving, by a central host computing device and from a plurality of wearable sensor devices, sensor signal data representative of biometric gait data of a subject and combining, by the central host computing device, the sensor signal data from each of the wearable sensor devices to generate a gait data collection in real-time. The method further includes providing, by the central host computing device, the gait data collection to a cloud-based biometric prediction engine and processing, by the biofeedback prediction engine, the gait data collection to generate vertical ground reaction force (vGRF) biofeedback prediction data associated with the subject in real-time.

According to another aspect of the method described herein, the wearable sensor devices include one wearable sensor to be mounted on a waist of the subject and two wearable sensor devices configured to be mounted on either ankle of the subject.

According to another aspect of the method described herein, the wearable sensor device configured to be mounted on the waist includes an accelerometer.

According to another aspect of the method described herein, the wearable sensor devices include wearable sensor devices configured to be mounted on thighs or feet of the subject.

According to another aspect of the method described herein, the gait collection data includes at least one of motion data or bilateral pressure data.

According to another aspect of the method described herein, the biofeedback prediction engine is executed by a machine learning algorithm.

According to another aspect of the method described herein, the plurality of wearable sensor devices includes at least one of: a motion sensor device, a force measurement sensor device, an accelerometer device, an inertial measurement unit (IMU) device, or a plantar pressure sensor device.

According to another aspect of the method described herein, the vGRF biofeedback prediction data is provided to the central host computing device for visual display on a user interface in real-time.

According to another aspect of the method described herein, the vGRF biofeedback prediction data includes a visual representation of one or more threshold levels of ground reaction force that compels the subject to alter step-by-step gait biomechanics in real-time.

According to another aspect of the method described herein, the vGRF biofeedback prediction data is provided to the central host computing device for auditory notifications on a user interface in real-time.

According to another aspect of the disclosed subject matter described herein, a system for detecting and monitoring gait using a portable gait biofeedback system includes a plurality of wearable sensor devices applied to a subject and configured to monitor for biometric gait data of the subject and generate sensor signal data representative of the biometric gait data. The system further includes a central host computing device configured for receiving, from the plurality of wearable sensor devices, the sensor signal data and combining the sensor signal data from each of the wearable sensor devices to generate a gait data collection in real-time. The system also includes a biofeedback prediction engine configured to receive the gait data collection from the host central computing device and to process the gait data collection to generate vertical ground reaction force (vGRF) biofeedback prediction data associated with the subject in real-time.

According to another aspect of the system described herein, the wearable sensor devices include one wearable sensor to be mounted on a waist of the subject and two wearable sensor devices configured to be mounted on either ankle of the subject.

According to another aspect of the system described herein, the wearable sensor device configured to be mounted on the waist includes an accelerometer.

According to another aspect of the system described herein, the wearable sensor devices include wearable sensor devices configured to be mounted on thighs or feet of the subject.

According to another aspect of the system described herein, the gait collection data includes at least one of motion data or bilateral pressure data.

According to another aspect of the system described herein, the biofeedback prediction engine is executed by a machine learning algorithm.

According to another aspect of the system described herein, the plurality of wearable sensor devices includes at least one of: a motion sensor device, a force measurement sensor device, an accelerometer device, an inertial measurement unit (IMU) device, or a plantar pressure sensor device.

According to another aspect of the system described herein, the vGRF biofeedback prediction data is provided to the central host computing device for visual display on a user interface in real-time.

According to another aspect of the system described herein, the vGRF biofeedback prediction data includes a visual representation of one or more threshold levels of ground reaction force that compels the subject to alter step-by-step gait biomechanics in real-time.

According to another aspect of the system described herein, the vGRF biofeedback prediction data is provided to the central host computing device for auditory notifications on a user interface in real-time.

According to another aspect of the disclosed subject matter described herein, one or more non-transitory computer readable media having stored thereon executable instructions that when executed by at least one processor of a computer cause the computer to perform steps including receiving, by a central host computing device and from a plurality of wearable sensor devices, sensor signal data representative of biometric gait data of a subject. The steps further include combining, by the central host computing device, the sensor signal data from each of the wearable sensor devices to generate a gait data collection in real-time. The steps further include providing, by the central host computing device, the gait data collection to a cloud-based biometric prediction engine. The steps further include processing, by the biofeedback prediction engine, the gait data collection to generate vertical ground reaction force (vGRF) biofeedback prediction data associated with the subject in real-time.

According to another aspect of the one or more non-transitory computer readable media described herein, the wearable sensor devices include one wearable sensor device configured to be mounted on a waist of the subject and two wearable sensor device configured to be mounted on either ankle of the subject.

The subject matter described herein may be implemented in hardware, software, firmware, or any combination thereof. As such, the terms “function” “node” or “engine” as used herein refer to hardware, which may also include software and/or firmware components, for implementing the feature being described. In one example implementation, the subject matter described herein may be implemented using one or more computer readable media having stored thereon computer executable instructions that when executed by the processor of a computer control the computer to perform steps. Example computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

FIG. 1 shows an example portable gait biofeedback system;

FIG. 2 shows an example deployment of wearable sensor devices on a subject's legs;

FIG. 3 shows an example portable gait biofeedback system;

FIG. 4A shows a front view of a wearable sensor device in an enclosure body including an attached battery;

FIG. 4B shows a wearable sensor device attached to a subject's shank by a strap;

FIG. 4C shows an example enclosure body;

FIG. 4D shows an example enclosure lid;

FIG. 5 shows example wearable sensor devices;

FIG. 6A shows an enlarged front view of a wearable sensor device;

FIG. 6B shows a left view of a wearable sensor device,

FIG. 6C shows a bottom view of a wearable sensor device,

FIG. 6D shows a right view of a wearable sensor device,

FIG. 7 shows a block diagram for an individual node of an example IMU-WBAN system;

FIG. 8 shows a graph with representative output of simultaneous recordings from three nodes located on the foot, tibia, and sternum capturing walking activity using all three IMU sensing modalities;

FIG. 9 shows a graph with representative output of gait change in response to overloading and underloading conditions, captured using magnetometer (top, tibia, Z-axis), gyroscope (upper-mid, sternum, Y-axis), and accelerometer (lower-mid, sternum, Y-axis) channels. Individual steps are easily identified from IMU data (upper right inset). Captured data is comparable to data collected from an equivalent commercial sensor (bottom);

FIG. 10A shows a graph of stance phase loading comparing measurements from in-sole sensors and force-sensing treadmills;

FIG. 10B shows a graph of loading peaks comparing measurements from in-sole sensors and force-sensing treadmills;

FIG. 11 shows a chart of vGRF measurements along a percent gait cycle in a control condition and a chart of vGRF measurements along a percent gait cycle in loading conditions;

FIG. 12 shows a subject viewing a visual display of real-time biofeedback while walking;

FIG. 13 shows a schematic diagram of analysis steps during an example test setup;

FIG. 14A shows a graph of a target vGRF signal in relation to BW over a gait cycle;

FIG. 14B shows a graph of a measured normal acceleration at the waist over a gait cycle;

FIG. 14C shows a graph of a measured normal acceleration at the shank over a gait cycle;

FIG. 14D shows a graph of a measured normal acceleration at the foot over a gait cycle;

FIG. 15A shows a graph of the measured vGRF signal in relation to BW over a gait cycle;

FIG. 15B shows a graph of foot, shank, and waist estimates of vGRF in relation to BW over a gait cycle;

FIG. 15C shows a graph of foot and waist estimates of vGRF in relation to BW over a gait cycle;

FIG. 15D shows a graph of shank and waist estimates of vGRF in relation to BW over a gait cycle;

FIG. 15E shows a graph of foot and shank estimates of vGRF in relation to BW over a gait cycle;

FIG. 15F shows a graph of shank estimates of vGRF in relation to BW over a gait cycle;

FIG. 15G shows a graph of foot estimates of vGRF in relation to BW over a gait cycle;

FIG. 15H shows a graph of waist estimates of vGRF in relation to BW over a gait cycle;

FIG. 16A shows a graph of MAE between measured and estimated vGRFs over the gait cycle;

FIG. 16B shows a graph of MAE between measured and estimated vGRFs at the instant of the loading peak;

FIG. 17A shows a graph of measured vGRFs for the Waist model;

FIG. 17B shows a graph of gait cycle MAEs for the Waist model;

FIG. 17C shows a graph of loading peaks for the Waist model;

FIG. 17D shows a graph of loading peak MAEs for the Waist model;

FIG. 17E shows a graph of measured vGRFs for the Foot-Waist model;

FIG. 17F shows a graph of gait cycle MAEs for the Foot-Waist model:

FIG. 17G shows a graph of loading peaks for the Foot-Waist model;

FIG. 17H shows a graph of loading peak MAEs for the Foot-Waist model;

FIG. 17I shows a graph of measured vGRFs for the Shank-Waist model:

FIG. 17J shows a graph of gait cycle MAEs for the Shank-Waist model:

FIG. 17K shows a graph of loading peaks for the Shank-Waist model;

FIG. 17L shows a graph of loading peak MAEs for the Shank-Waist model;

FIG. 18 shows a graph comparing weighted acceleration and estimated vGRF;

FIG. 19A show an example visual displays that includes visual biofeedback:

FIG. 19B show an example visual displays that includes visual biofeedback;

FIG. 19C show an example visual displays that includes visual biofeedback:

FIG. 20 shows an example visual display summarizing a walking session by a subject on a personal computer;

FIG. 21 shows an example visual display on a personal computer;

FIG. 22 shows example footfall pressure sensors;

FIG. 23 is a flow chart illustrating an example clinician workflow on a user interface;

FIG. 24 is a flow chart illustrating an example consumer workflow on a user interface; and

FIG. 25 is a flow diagram illustrating an example method for detecting and monitoring gait using a portable gait biofeedback system.

### DETAILED DESCRIPTION

The subject matter described herein relates to methods, systems, and computer readable media related to a portable gait biofeedback system for rehabilitating aberrant gait. Limb-level biomechanical outcomes are linked to joint tissue and symptom changes related to osteoarthritis onset and progression. Specifically, lower first peak vGRF, in particular, associates with more deleterious biological joint tissue changes and worse patient-reported outcomes that are consistent with knee osteoarthritis development and progression. Altering peak vGRF magnitudes via real-time biofeedback may normalize limb-level gait biomechanics and limit the biological changes leading to osteoarthritis development.

In some aspects, the disclosed subject matter is designed in part to meet the critical and immediate need to reestablish and maintain normal walking gait biomechanics. The disclosed system is further designed for the purpose of optimizing joint tissue loading to prevent the development and/or progression of chronic musculoskeletal conditions. Moreover, the disclosed system can be utilized to accelerate patient recovery following a joint injury and/or surgery. Using a comprehensive scientific approach, a specific biomechanical variable (i.e., peak vertical ground reaction force (vGRF) in early stance) that is associated with biological and patient-reported changes consistent with chronic knee joint breakdown (e.g., osteoarthritis development) has been identified.

As such, a portable gait biofeedback system capable of detecting, monitoring, and treating aberrant GRF during walking is described herein. Notably, the disclosed system can be deployed in real-world environments for use by rehabilitation clinicians to treat patients with musculoskeletal disorders or by patients via a direct-to-consumer model. In some embodiments, the portable gait biofeedback system comprises a plurality of wearable sensors that are applied directly to a patient user, a software collection component (that is executed by a hardware computing device) that is configured to monitor and collect the sensor data from each of the plurality of sensors, and a cloud-based AI data analysis engine. For example, the plurality of wearable sensors can be applied to a patient user and configured to measure vGRF data that is experienced by the patient's feet and legs. The measured data is then wirelessly sent to the software collection component, which in turn forwards the data to the AI data analysis engine in the cloud. The data analysis engine is adapted to process the vGRF data to predict step-to-step vGRF values. The data analysis engine may subsequently display the predicted data via real-time biofeedback training modes designed to optimize lower extremity loading.

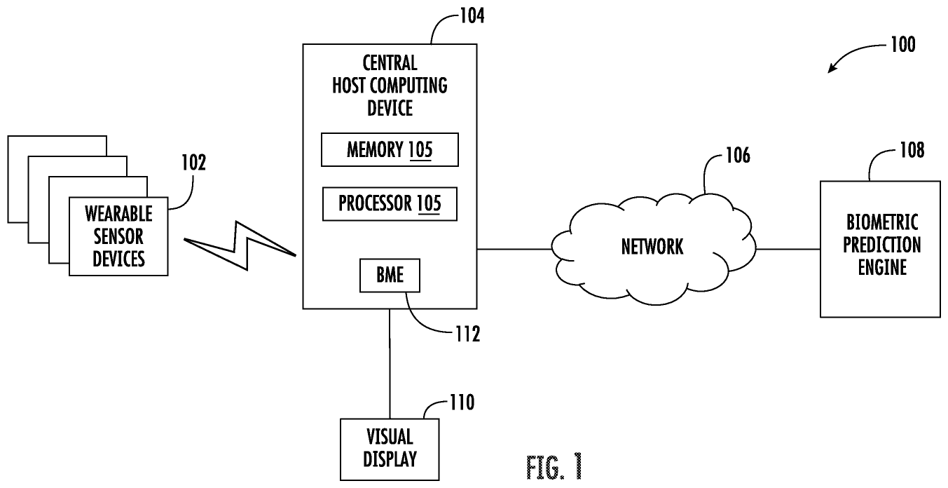

FIG. 1 illustrates an exemplary portable gait biofeedback system 100 that includes a plurality of wearable sensor devices 102, a central host computing device 104, a cloud-based biometric prediction engine 108, and a visual display device 110. Central host computing device 104 can include a memory 105 and at least on hardware processor 107 configured to perform the steps described herein. In some embodiments, wearable sensor devices 102 may include a series of eight (8) custom inertial measurement units (IMUs) configured to communicate directly with central host computing device 104 and provide sensor signal data that is representative of biometric gait data of a patient or subject. In some embodiments, system 100 can utilize fewer sensor devices 102 that are adapted and/or trained to provide the same predicting accuracy. In some embodiments, wearable sensor devices 102 may also be configured to integrate with an optional plantar pressure circuit that is adapted to record bilateral pressure from under the subject's feet.

In other embodiments, sensor devices 102 may be configured to communicate with central host computing device 104 (e.g., personal computer, laptop, tablet, or mobile device) via a central hub transceiver device or “base station” (shown in FIG. 3). In particular, the central hub transceiver device or base station may receive sensor signal data from the eight IMU devices (e.g., via 8-1 communication) and subsequently forward the received sensor signal data to central host computing device 104, which may be configured to operate as a system host.

In some embodiments, central host computing device 104 may include a biofeedback management engine 112 that is configured to establish a data communication pipeline with wearable sensor devices 102. Notably, the data communication pipeline can be used by biofeedback management engine 112 to monitor and accumulate sensor signal data from wearable sensor devices 102. Real-time processing may be established between biofeedback management engine 112 and each of wearable sensor devices 102 (e.g., wirelessly request or ping for sensor signal streams from each of the sensor devices and process the same). In some embodiments, biofeedback management engine 112 may also comprise a data analytics engine or software algorithm/component that is stored in memory and executed by hardware processor 107 of central host computing device 104.

After the sensor signal data is received from wearable sensor devices 102, central host computing device 104 may be configured to collect and combine the sensor signal data to generate a collection of gait data (i.e., “gait data collection”). Notably, the collection, combination, and associated processing of the sensor signal data by the central host computing device 104 can be conducted in real-time or in near real-time.

Once generated, the collection of gait data is forwarded by central host computing device 104 to a biometric prediction engine 108 via a communications network 106 (e.g., the Internet). The biometric prediction engine 108 can be any data analytics software component that is stored in memory and executed by a hardware processor associated with one or more cloud-based computing devices (e.g., Amazon Web Services servers). In some embodiments, biometric prediction engine 108 may include a machine learning algorithm and/or artificial neural network (ANN) that has been trained to process the gait data collection and generate vGRF biofeedback prediction data in real-time. For example, system 100 can include a trained machine learning algorithm (e.g., supported by a remote hardware based host) that is capable of predicting step-to-step vGRF values from the measured sensor signal data received from the local biofeedback management engine 112 on the central host computing device 104.

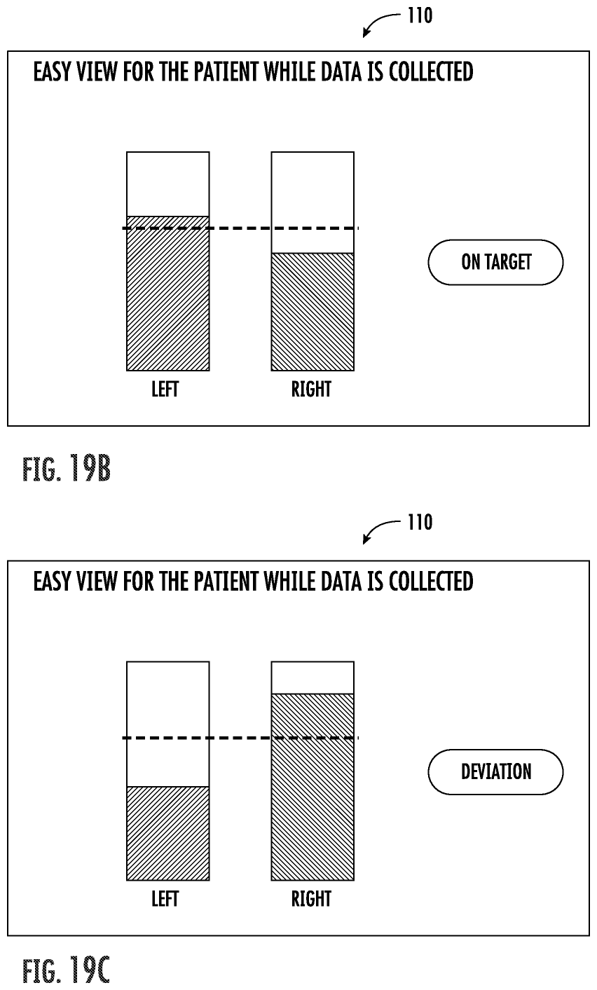

Further, the generated vGRF biofeedback prediction data may be sent by biometric prediction engine 108 to central host computing device 104 in real-time for user access. For example, the biofeedback prediction data can be presented to a subject (e.g., patient) or other user (e.g., physician) via a visual display 110 associated with central host computing device 104. In some embodiments, visual display 110 can be tablet-based or PC-based. Visual display 110 can include a graphical user interface (GUI). Visual display 110 may include one or more custom user interfaces in which the user and/or a treating physician may utilize to engage with the portable gait biofeedback system in several operating modes (e.g., detect, monitor, and/or treat). These user interfaces may be modeled after other successful laboratory-based gait biofeedback systems. Example user interfaces associated with the disclosed gait biofeedback system are presented in FIG. 12, FIGS. 19A-19C, FIG. 20, and FIG. 21. As shown in FIG. 12, visual display 110 can present visual biofeedback in real-time of the generated vGRF biofeedback prediction data for the subject to view while walking. The visual biofeedback can include a target vGRF, allowing the subject to compare the current vGRFs on the right and left foot to what the vGRF should be. This provides the subject with real-time feedback to self-correct as well as recognize how a healthy gait feels. The vGRF biofeedback prediction data can be provided to the central host computing device for auditory notifications on a user interface in real-time. Auditory notifications can include notifications of current vGRF predictions for the right and left foot and/or a comparison between the current vGRF predictions and threshold levels for vGRF, for example accuracy percentages.

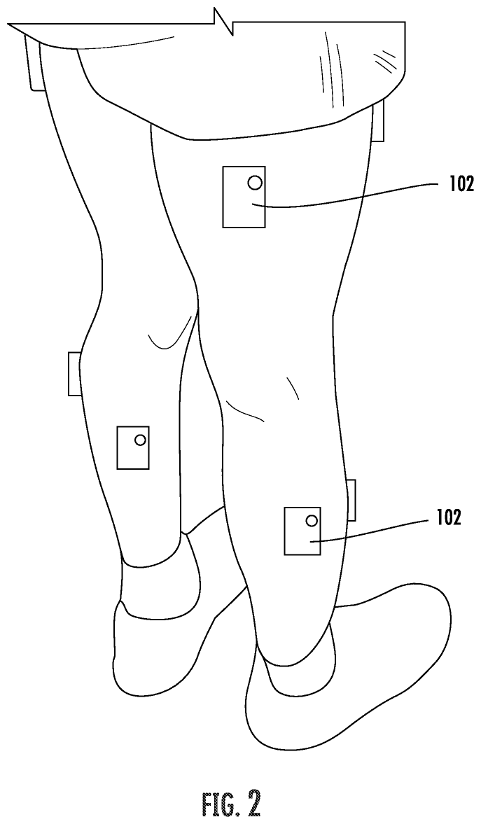

FIG. 2 shows an example deployment of wearable sensor devices 102 on a subject's legs. Wearable sensor devices 102 can be positioned on various places on a subject's body. For example, in an aspect of the described subject matter, three wearable sensor devices 102 can be positioned on a subject's body, including one wearable sensor device 102 on the subject's waist, and one wearable sensor device 102 on each ankle of the subject. One or more wearable sensor devices 102 can be positioned on a subject, including on the waist, shank such as calf and/or shin, thigh, foot, sternum, and the like. Wearable sensor devices 102 can each include an accelerometer, a gyroscope, and/or a magnetometer. As a nonlimiting example, system 100 can include a first wearable sensor device 102 configured to be mounted on a waist and second and third wearable sensor devices 102 configured to be mounted on either ankle, and the wearable sensor devices 102 may each include at least an accelerometer. The wearable sensor devices 102 may also include gyroscopes and/or magnetometer. In another aspect, additional wearable sensor devices 102 may be configured to be worn on thighs and/or feet.

### Wireless Body Area Network for Clinical Gait Rehabilitation

FIG. 3 shows an example portable gait biofeedback system 300. Wireless body area networks (WBANs) are composed of sensor nodes (e.g., wearable sensor devices 102) worn across multiple locations on the body of a human subject 302, which are frequently employed in the area of digital health monitoring. Wearable sensor devices 102 each include at least one sensor, such as an Inertial Measurement Unit (IMU). An IMU is a low-cost microelectromechanical system and one type of sensor device that is capable of quantifying motion in terms of linear acceleration, angular rotation, and orientation within an external magnetic field. Wearable IMUs incorporating sensors along three spatial axes enable on-body motion tracking, providing for the collection of motion data required for complex gait monitoring, and potentially allowing these capabilities to be translated from specialized lab environments to the clinic and beyond. Clinical studies have established agreement between motion data collected by inertial sensors and force plate measurements in patient populations with significant musculoskeletal damage.

Current efforts which deploy IMUs for gait analysis rely on commercial systems, such as the Delsys Trigno Wireless Biofeedback System, to estimate body orientation within 3D space. Notably, this system is prohibitively expensive, and its closed-source design prevents modification for experimental requirements, e.g., providing raw, unfiltered data, or the incorporation of additional sensor inputs and timing signals. As a result, this multi-node IMU system is currently impractical for widespread use in the collection of high-resolution limb position data.

In contrast, an example aspect of the described system includes a novel 8:1 IMU-WBAN that has been developed and evaluated in the context of real-time gait monitoring. This system allows for rapid and high-resolution sampling of accelerometer, gyroscope, and magnetometer data from a plurality of points (e.g., 8 points) on the body simultaneously, which can be analyzed to identify gait abnormalities and prompt any necessary interventions. The disclosed system can enable gait monitoring in a variety of settings, and is designed for affordability, wearability/ease of use, and low power consumption. Herein, examples of hardware design, communication protocol implementation, device enclosure design, human subject testing, and targets of future work are presented. It is understood that the disclosed subject matter is not limited to implementing eight nodes, but may include 3, 4, 5, 6 7, 9, or more nodes (e.g., wearable sensor devices 102) on subject 302.

### System Design

In some aspects of the disclosed subject matter, the system is configured to utilize wearable sensor devices 102, such as IMUs, to measure vGRF and requires highly resolved, subsecond (10-1000 Hz) recording of limb position and orientation data across multiple sites on the lower body, used later to estimate limb loading data across multiple sites on the lower body. To facilitate this data acquisition, an example WBAN comprising of 8 peripheral nodes was designed. Notably, these nodes (e.g., wearable sensor devices 102) may be wirelessly connected to a single off-body base station 304 and interfaced with a host computer, such as central host computing device 104.

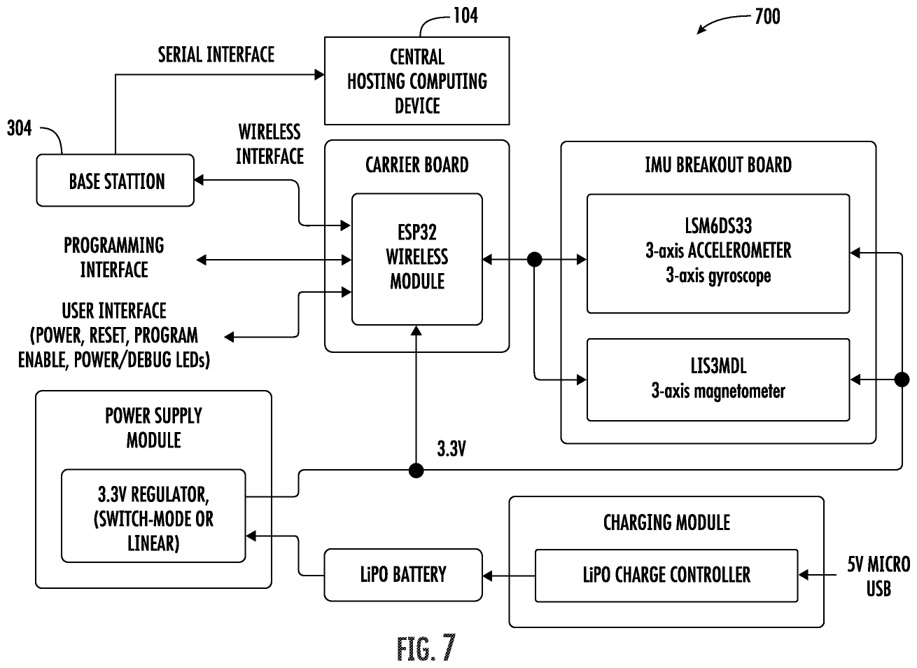

FIG. 7 shows a block diagram 700 for an individual node of an example IMU-WBAN system. In some aspects of the disclosed subject matter, each node employs a 9-axis IMU, a wireless microcontroller unit (ESP32-WROOM-32D, Espressif Systems), and supporting electronics. To provide for expansion of device capabilities as end-user requirements change, nodes can be modular in design, i.e., IMU and power management subcircuits are self-contained on separate printed circuit boards (PCBs) soldered together using castellated vias and a standardized inter-board pinout.

In some aspects of the disclosed subject matter, wearable sensor devices 102 are powered by a 400 mAh lithium polymer (LiPO) battery, providing a minimum 2.85 hour operational lifetime, which can be charged in situ by an optional charge controller module (MCP73831, Microchip Technologies) receiving a 5V input through a micro USB connector. A regulated 3.3V bus is maintained using one of two power supply modules, incorporating a buck-boost switch mode regulator (ADP2503, Analog Devices) or a low-dropout linear regulator (ST1L05, STMicroelectronics), respectively. For devices tested on-body, the former supply module was used to maximize battery lifetime. In some embodiments, communication with the IMU module may be accomplished using the I2C protocol.

In some aspects of the disclosed subject matter, each IMU contains a 3-axis accelerometer and 3-axis gyroscope (e.g., LSM6DS33, STMicroelectronics) and a 3-axis magnetometer (e.g. LIS3MDL, STMicroelectronics) that collectively provide 9 axes of data, each with 16-bit resolution. A user interface can facilitate external device management, providing for power toggling, system reset, enabling flash programming, and providing status LEDs for system power, battery charge status, and debugging. An additional analog-to-digital converter (ADC) input may be reserved for use with a footfall pressure sensor.

During operation, the base station may establish communications with all available nodes within its designated cluster, as determined by a set of hard-coded media access control (MAC) addresses. Using the proprietary ESPNow protocol, an implementation of the IEEE 802.11 2.4 GHz standard, the base station can sequentially send a ping to each node. Connected nodes respond with a 44-byte data packet, containing identifying delimiting header and footer byte pairs, node ID and status information, and 4 bytes each of the most recent accelerometer, gyroscope, and magnetometer data from the IMU module. New sensor data is polled continuously by the node until a ping is received, with packet transfers occurring at a minimum rate of 20 Hz. Upon receipt by the base station, each 44-byte packet is immediately transferred to the host computer over a serial connection for data logging and real-time display. Individual data streams, i.e., linear and rotational motion and displacement within an external geomagnetic field, are combined and synthesized by an application on the host computer to provide insight on subject posture and gait.

### Enclosure Design

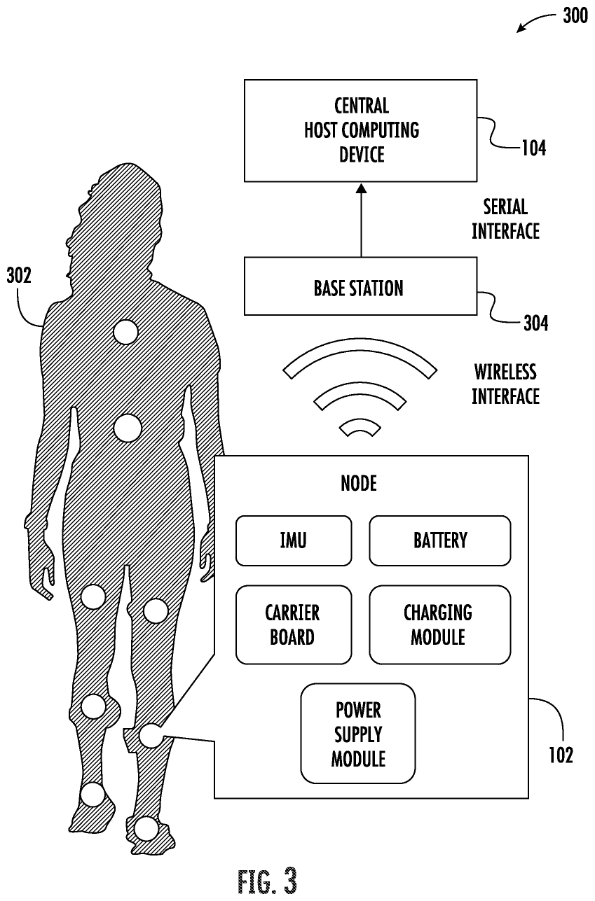

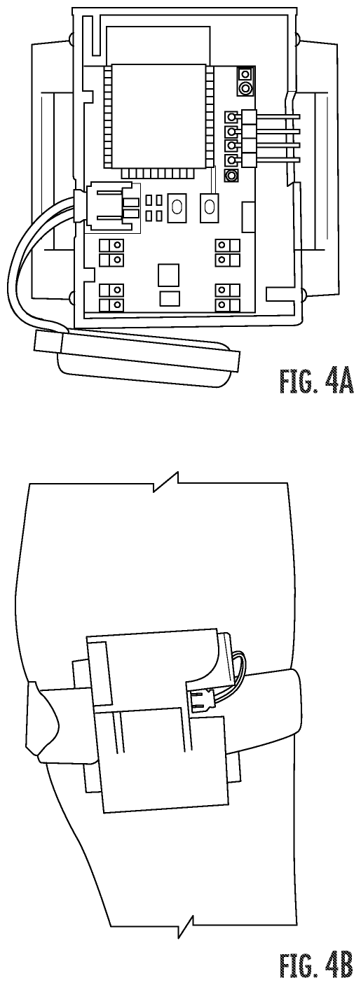

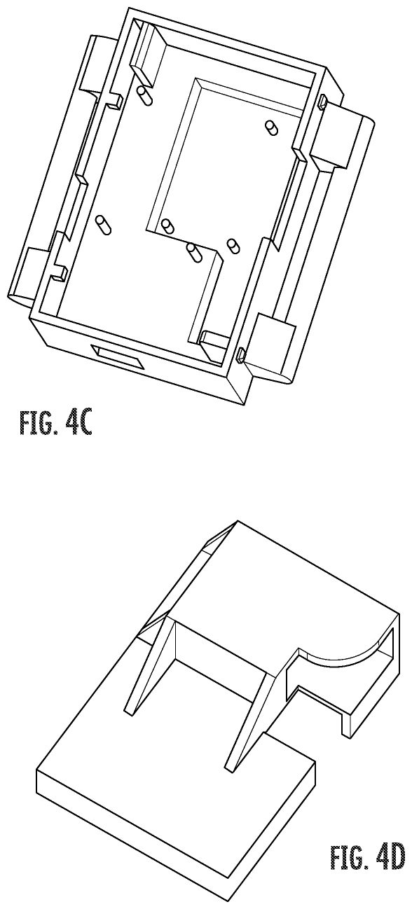

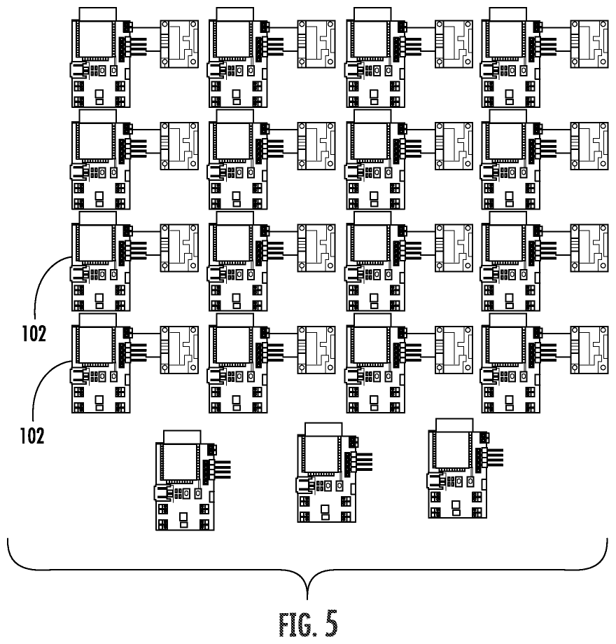

FIGS. 4A-4D show components of wearable sensor device 102. In some aspects of the described subject matter, wearable sensor devices 102 can each include an enclosure body holding the IMU, a carrier board, and charging modules. The encloser can include an enclosure body 402 and a removably attachable enclosure lid 404. FIG. 4A shows a front view of wearable sensor device 102 in enclosure body 402 including an attached battery 403. Wearable sensor device 102 is without enclosure lid 404. Enclosure body 402 can include handles on either side to accommodate hook-and-loop or elastic straps for attachment to the patient. FIG. 4B shows wearable sensor device 102 attached to subject's 302 shank by a strap. FIG. 4C shows enclosure body 402. The inside of the enclosure body 402 is designed to minimize movement of the IMU and carrier board and eliminate noise originating from spurious motion. The inside of enclosure body 402 can include recesses shaped by fit components of wearable sensor device 102. The IMU and carrier board can be held in place using posts which tightly interface with mounting holes present both components. FIG. 4D shows enclosure lid 404. Enclosure lid 404 can be configured to lock in place using printed tabs and receivers. Enclosure lid 404 may also feature a reinforced compartment for battery storage. Enclosure lid 404 may include one or more opening to accommodate components of wearable sensor device 102 that may extend from the wearable sensor device 102 and/or for easy coupling to an external source, such as a power supply. In some aspects of the described subject matter, enclosure lid 404, when attached to enclosure body 402, can form a waterproof seal. In one aspect, the IMU enclosure is designed in SOLIDWORKS (Dassault Systèmes) and printed using a Form 3+ resin 3D printer (Formlabs) in photopolymer resin (Black v4, Formlabs). In other aspects, the enclosure lid and enclosure body may be permanently sealed. FIG. 5 shows example wearable sensor devices 102.

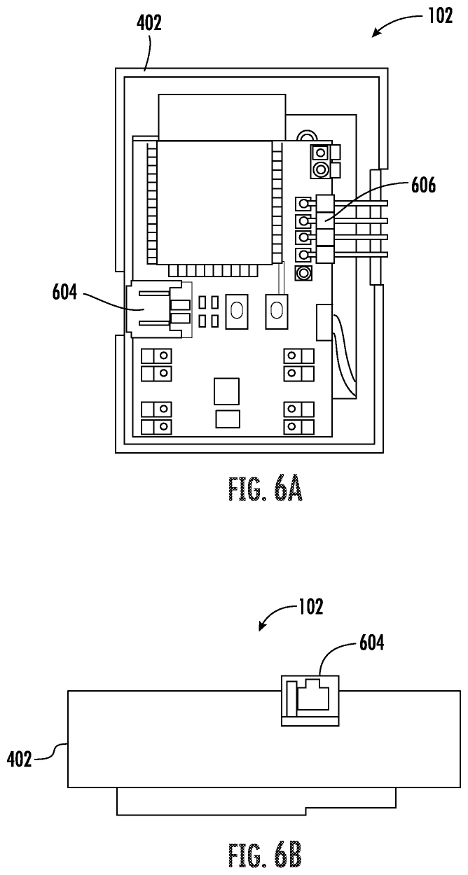

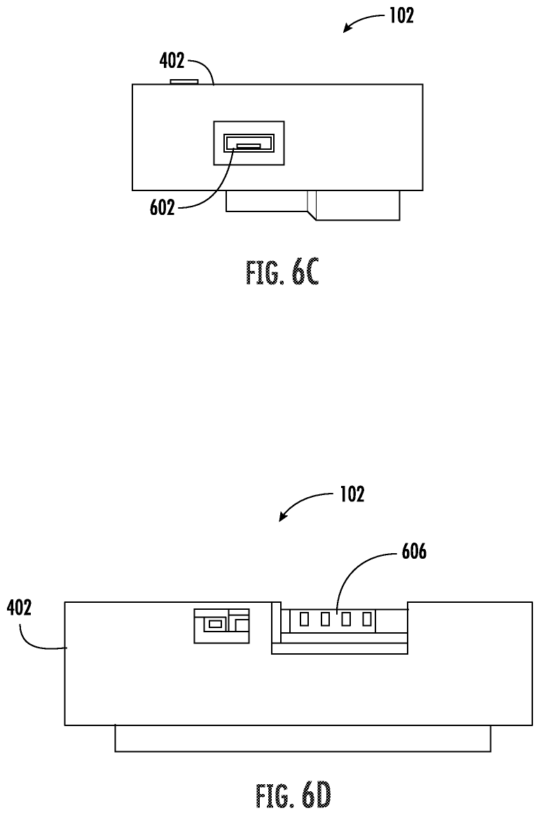

FIGS. 6A-6D show wearable sensor device 102 in enclosure body 402. Enclosure body 402 can feature openings for a charging port 602, for example without limitation a micro-USB charging port, a JST-PH battery connector 604, and/or pin headers 606 for the MCU programming interface. FIG. 6A shows an enlarged front view of wearable sensor device 102. FIG. 6B shows a left view of wearable sensor device 102. FIG. 6C shows a bottom view of wearable sensor device 102. FIG. 6D shows a left view of wearable sensor device 102. The underside of the enclosure may have a raised flat plane for mounting of different skin-safe adhesives, if desired.

The following are examples of tests conducted using one or more aspects of the disclosed subject matter.

### Human Subject Testing

All human subject tests were conducted in accordance with approved protocols (UNC IRB #21-1693). Participants engaged in a single-visit observational study and walked at approximately 1.2 m/s on a split-belt force-measuring treadmill, while monitored with wearable inertial sensors. For these tests, 16 sensors were used: 8 commercially-available inertial sensors (Delsys Trigno Wireless System), and the developed 8:1 IMU-WBAN. Closely-spaced sensor pairs (one of each type) were mounted about the lower body: on both insteps, the lateral surfaces of both thighs, the distal, anterior surfaces of each tibia, the upper sternum, and the lumbar spine. Double-sided tape was used to affix the Delsys sensors to the subject. WBAN nodes were affixed by silicone adhesive (Upright, Tel Aviv, Israel). All nodes, except those placed on the lower sternum, were held by Velcro® straps.

Participants walked for approximately 4 minutes (˜400 steps) to acclimate to the task and generate baseline vGRF data. Next, participants walked 4 minutes under two different loading conditions: 5% increased step force and 5% decreased step force, as compared to baseline vGRF. vGRF was systematically manipulated using real-time gait biofeedback, displayed on a screen in front of the participant. Measured vGRF was displayed along with a 5% increased or decreased target vGRF. These increased and decreased loading conditions were used to evaluate sensor detection of changes in gait biomechanics.

### Device Design

Critical performance metrics include sampling rate, resolution, number of axes, and range. Significant improvements were achieved compared to commercially-available, expensive, proprietary systems, Comparisons to the Delsys Trigno Wireless IMU System, an established clinical standard for inertial limb monitoring, are summarized in Table 1 below. The developed WBAN can be open-source, enabling access to raw output data, in contrast with existing commercial systems which preprocess all generated output data. Prioritization of open-source design also enables optional integration of additional sensor inputs and interfaces, e.g., a footfall pressure sensor.

TABLE 1
System Performance Comparison

| | | |
|---|---|---|
| Specification | This Work | Delsys Trigno |

| System Cost | ≤$487.48 | | $12,909 | |
|---|---|---|---|---|
| Battery Life | ≥2.85 | hours | 4 | hours |
| Sampling Rate | ≥20 | Hz | 2000 | Hz |
| Size | 55 × 39 × 30 | mm | 34 × 25 × 12 | mm |
| Range | ≥160 | m | 20 | m |

| | | | |
|---|---|---|---|
| Weight | 11.5 g (peripheral) | 14.7 | g |
| | 48.0 g (enclosed) | | |

| | | |
|---|---|---|
| Axes | 9 | 9 |
| Resolution | 16 bits per axis | 16 bits per axis |
| Communication | ESPNow | BLE |
| Documentation | Open source | Closed source |
| Additional Features | ADC input | EMG |

One key advantage for broad distribution and use of the reported WBAN is cost per system. An 8-sensor Trigno system is priced at $12,909, while the reported WBAN costs $56.46 for a single node or $487.48 for 8 peripheral devices and one base station, as prototyped. This 8:1 WBAN achieved an effective data transfer rate in excess of 20 Hz per node. The resolution and battery life achieved in this design are comparable to those observed in the Trigno system. In its current iteration, the 8:1 IMU-WBAN is larger and heavier than the Trigno devices, due in part to modular hardware design employed in this initial revision.

### Real-Time Gait Adjustment Data

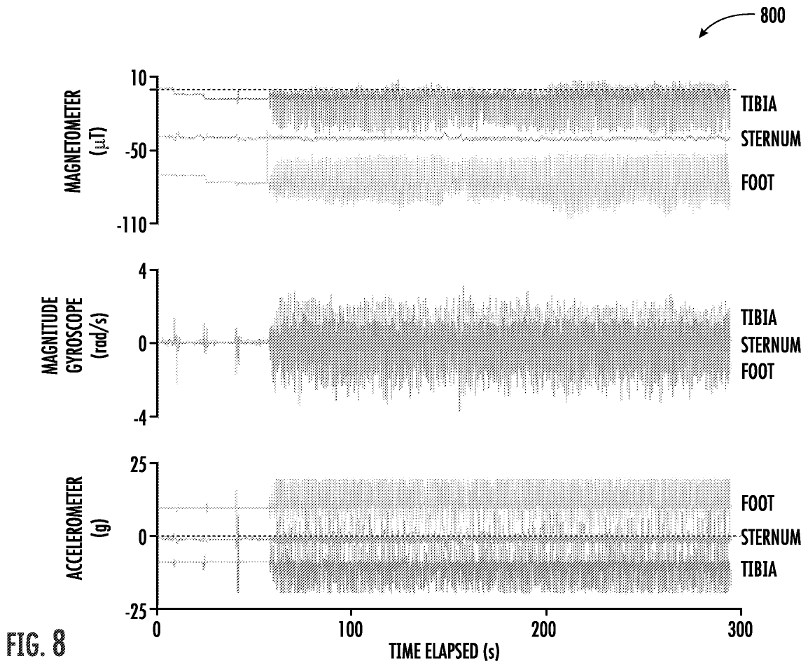

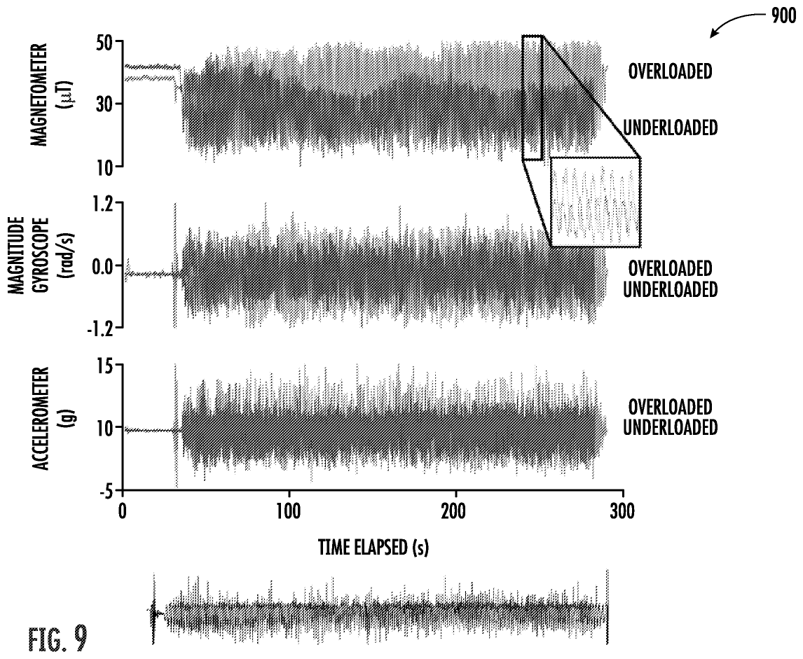

The 8:1 IMU-WBAN was evaluated in a biofeedback test, which required human subjects to walk normally before attempting to overload or underload their vGRF to 5% higher or lower than their baseline. This procedure was chosen as a proof of concept to validate the 8:1 IMU-WBAN response to minimal variations in gait. FIG. 8 shows a graph 800 with representative output of simultaneous recordings from three nodes located on the foot, tibia, and sternum capturing walking activity using all three IMU sensing modalities. Referring to FIG. 8, across multiple locations, the 8:1 IMU-WBAN exhibited successful detection of changes in gait mechanics resulting from test subjects' intentional modulation of vGRF. FIG. 9 shows a graph 900 with data at three different nodes under two loading conditions (i.e., overload and underload) collected sequentially using all three IMU sensing modalities. Overload conditions consistently produced a sensor response of higher magnitude than both control and underload conditions. These initial data serve as a proof of concept for the system's ability to track limb movement with high fidelity.

### Conclusions

The design and preliminary characterization of an 8:1 IMU-WBAN for motion tracking and vGRF quantification was reported. The 8:1 WBAN overcomes the fundamental limitations of standard 3-D imaging and force table-based gait diagnostic systems by providing raw data across 9 axes via accelerometers, magnetometers, and gyroscopes. This product provides significantly improved accessibility when compared to currently available IMU systems, using commercial off the shelf hardware and software to enable access to raw data, which is pertinent for future integration into machine learning algorithms for clinical gait therapy. Additional notable features include significant cost reduction and vastly improved system operating range. Proof of concept for the 8:1 IMU-WBAN was established through a vGRF biofeedback protocol, in which subjects' efforts to modify their gait, in both overload and underload conditions, were captured holistically.

Sensor parameters can be tuned to allow for a wider dynamic range across all modalities. The peripheral ADC input can be used to integrate a flexible pressure sensing node under the heel to enable the quantification of the patient's gait condition and direct load-bearing tendencies. The 8:1 IMU-WBAN can be used to feed machine learning algorithms for live gait correction therapy via biofeedback.

Using a sophisticated laboratory setup, the disclosed subject matter can cue individuals to change peak vGRF using the gait biofeedback system, which normalizes multiple critical biomechanical components of walking gait (e.g., knee flexion excursion and knee extension moment). This results in beneficial biological changes to joint tissues (i.e., decreased serum concentrations of cartilage oligomeric matrix protein, a biomarker of joint tissue breakdown). Further, extensive research has been conducted to determine the optimal vGRF threshold that can be used to clinically modify gait biomechanics for the purpose of decreasing osteoarthritis-related symptoms following lower-extremity joint injury. As such, the disclosed biofeedback system has the unprecedented ability to target and modify specific gait biomechanics that are linked to osteoarthritis development.

In some embodiments, the disclosed portable gait biofeedback system can be deployed in real-world environments for use by rehabilitation clinicians to treat patients with musculoskeletal disorders. Custom wearable sensor configurations and novel algorithms are utilized to detect and modify peak vGRF during gait and provide the patient or subject a real-time user interface that can be displayed on a portable screen or headset (e.g., visual display 110 in FIG. 1). A single portable gait biofeedback system can be used by multiple individuals with minimal setup time and can be effectively administered during treadmill or over ground walking. Currently, no other gait biofeedback system exists with the capability to detect and modify biomechanical determinants of joint loading to allow for evidence-based changes in gait biomechanics with the goal of maintaining long-term joint health. The disclosed system may allow clinicians to easily administer clinically-relevant and effective gait biofeedback to patients in a time-effective manner. Overall, the disclosed portable gait biofeedback system can revolutionize treatment of aberrant gait biomechanics associated with the development of various musculoskeletal conditions (e.g., knee osteoarthritis) by allowing clinicians to easily administer gait retraining in the clinic.

Efficacy of in-Sole Sensors to Detect Limb Loading Changes Using Biofeedback

As indicated above, the wearable sensor devices 102 shown in FIG. 1 may include and/or be integrated with optional plantar pressure circuits (e.g., in-sole sensor devices) to detect limb loading changes using biofeedback. Notably, clinicians currently lack precise, accessible, and evidence-based technology to detect and treat subtle, yet potentially harmful changes in walking biomechanics. For example, accurate, user-friendly, and reliable devices capable of measuring the most relevant outcomes of knee joint loading during walking could improve clinical outcomes by detecting aberrant biomechanics and integrating with biofeedback to optimize loading. Altered peak vertical ground reaction force (vGRF) during the impact phase of gait can be associated with biochemical markers indicative of joint tissue breakdown. Furthermore, modifying peak vGRF via biofeedback can elicit favorable changes in knee joint biomechanics, showing practicality for gait retraining in populations at risk for chronic knee dysfunction. However, detecting vGRF changes during walking and implementing biofeedback typically requires using laboratory-grade force-sensing treadmills. Commercially-available devices do not yet exist to provide these services to clinicians and patients seeking to mitigate risk of chronic pain and dysfunction. Accordingly, in-sole wearable sensors may provide an accessible option for: (i) estimating vGRF profiles during walking and (ii) driving real-time biofeedback to modify gait biomechanics and tissue biology. Notably, the disclosed system can be configured to utilize in-sole sensors (SS) as a sufficient alternative to force-sensing treadmills (FT) and to estimate changes in vGRF signals prescribed using real-time biofeedback. It is hypothesized that SS may provide a comparable vGRF estimate to that derived via FT, and that SS would be as sensitive as FT to biofeedback-induced changes.

### Methods

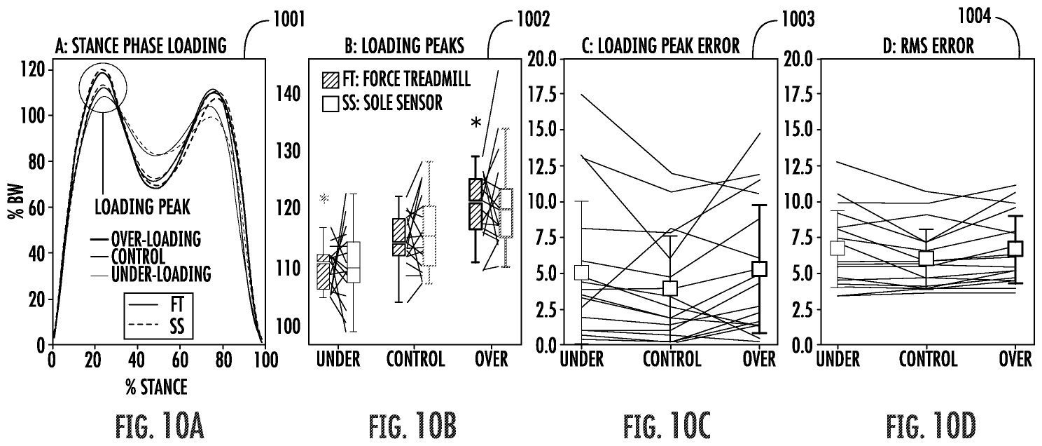

Now referring to FIGS. 10A-10D, we recruited a convenience sample of 17 participants (i.e., 10 female, age: 26.0±3.6 years, height: 1.74±0.08 m, mass 69.1±8.5 kg, preferred walking speed 1.35±0.14 m/s [mean±standard deviation]) to walk at their preferred speed for 5 minutes (Control) on a FT. We recorded their typical loading peak vertical ground reaction force from the FT for each leg during the last minute of the control trial. The participants then performed two 5-minute targeted biofeedback trials at +/−5% (Over/Under) of their typical loading peak vGRF measured from the FT (see graph 1001 in FIG. 10A). FIG. 10A shows a graph 1001 of stance phase loading comparing measurements from in-sole sensors and force-sensing treadmills. During biofeedback trials, participants saw their average loading peak vGRF for each side (left & right) from their previous 2 steps. During each trial, we recorded force platform kinetics (Qualisys Track Manager, Qualisys AB, Göteborg, Sweden) and bilateral in-shoe sole forces (LoadSol, Novel, Saint Paul, Minnesota, USA). After resampling the signals to a common 100 Hz, we synchronized the FT and SS signals using a cross-correlation function and extracted the peak loading force across both systems using a peak finding algorithm. We used a repeated measures ANOVA (with Tukey post-hoc) to test for differences between FT and SS and across biofeedback conditions (α=0.05), We also present (partial) eta square (η₍ₚ₎²) effect sizes.

### Results and Discussion

FIG. 10B shows a graph 1002 of loading peaks comparing measurements from in-sole sensors and force-sensing treadmills. Asterisks (*) indicate significant Tukey's post-hoc difference vs. control. It was found that a significant main effect for biofeedback condition (p<0.001, ηₚ^{2=0.713}, see graph 1002 in FIG. 10B). However, we found no main effect of measurement modality (FT vs. SS) nor an interaction between measurement modality and biofeedback condition (p≥0.312, ηₚ^{2≤0.057}). Loading peak vGRFs estimated via SS were similar to those from FT (p≥0.516, η²≤0.013, see graphs 1002-1004 in FIGS. 10B-10D). FIG. 10C shows a graph 1003 of loading peak error and FIG. 10D shows a graph 1004 of RMS error. However, only FT could distinguish between loading biofeedback conditions (e.g., SS under and over vs. control: p≥0.076, η2≤0.128; FT under & over vs. control: p≤0.017, η2≥0.193, see graph 1002 in FIG. 10B), On average, compared to FT, SS estimated loading peaks within ˜5% (see graph 1003 in FIG. 10C) and displayed 7% RMS error overall (see graph 1004 in FIG. 10D).

Genetic Fuzzy System for vGRF Prediction

In some aspects of the described subject matter, biometric prediction engine 108 (as shown in FIG. 1) may comprise a Genetic Fuzzy System for vGRF prediction. For example, the disclosed Genetic Fuzzy System (GFS) may be trained using the FuzzyBolt© framework. GA tunes the membership function boundaries and the rules. For this work, five membership functions are used to define each input and output variable.

In some aspects of the described subject matter, the GFS predicts the left vGRF and right vGRF at each instant based on the 26 sensor values at that instant. For example, the 26 input variables may include the left and right shank, foot and thigh acceleration, the sacrum acceleration, torso acceleration, and the left and right planar pressure values as percentage of body weight. The GFS can be configured to process these values based on the membership functions for each input and the rules.

In some aspects of the described subject matter, the underlying process of fuzzy system for evaluating the output based on the input variables can involve 3 main steps that can be executed by the biometric prediction engine 108: fuzzification, rule evaluation and defuzzification. Fuzzification involves converting the actual variable values to membership values based on the corresponding membership functions. These membership values are then used to evaluate each rule in the rulebase. The membership values corresponding to the antecedents in each IF-THEN rule are multiplied to obtain its rule firing value. The rule firing values are then multiplied with the corresponding output membership functions defined on the consequent side of each rule. These values can then be added to obtain a membership shape within the fuzzy domain. The x-coordinate of the centroid of this shape is considered as the output of the fuzzy system.

In some aspects of the described subject matter, the membership functions and the rules are defined during the training process. Proper identification of membership functions and rulebase allows for improved predictive performance. For the training of the GFS, an appropriate cost function is identified. Since we are interested in accurate predictions of higher vGRF values, the weight is defined as follows and will be used when evaluating the weighted RMSE.

$${w}_{ij} = \{ \begin{matrix}1 if {vGRF}_{ij} > 0.8 \\ 0.1 if {vGRF}_{ij} \leq 0.8\end{matrix}$$

where i refers to the index of the datapoint and j can be one of two values: 1 (for left) or 2 (for right).

The weighted RMSE, defined below, is considered as the cost function during training, where [symbol] is the predicted vGRF value. GA tunes the membership function and rule parameters to minimize the cost function as follows:

$$C = \frac{1}{2} {\sum }_{j = 1}^{2} \sqrt{\frac{{\sum }_{i = 1}^{N} \vert {{w}_{ij} ( {vGRF}_{ij} - )}^{2}}{{\sum }_{i} {w}_{ij}}}$$

Once trained, the GFS model is used to predict vGRF values using the 26 sensor values for new subjects.

### Effect of Sensor Number and Location on Accelerometry-Based Vertical Round Reaction Force Estimation During Walking

The optimal number of sensors and sensor placements for predicting accurate vGRF from accelerometry remains unknown. Our goals were to: 1) determine how many sensors and what sensor locations yielded the most accurate vGRF loading peak estimates during walking; and 2) characterize how prescribing different loading conditions affected vGRF loading peak estimates. We asked 20 young adult participants to wear 5 accelerometers on their waist, shanks, and feet and walk on a force-instrumented treadmill during control and targeted biofeedback conditions prompting 5% underloading and overloading vGRFs. We trained and tested machine learning models to estimate vGRF from the various sensor accelerometer inputs and identified which combinations were most accurate.

We found that a neural network using one accelerometer at the waist yielded the most accurate loading peak vGRF estimates during walking, with errors around 4.3% body weight. The waist-only configuration was able to distinguish between control and overloading conditions prescribed using biofeedback, matching measured vGRF outcomes. Including foot or shank acceleration signals in the model reduced accuracy, particularly for the overloading condition. Our results suggest that a system designed to monitor changes in walking vGRF or to deploy targeted biofeedback may only need a single accelerometer located at the waist.

Cost-effective wearable sensor solutions may provide a clinically-feasible option to monitor limb loading. For example, accelerometers and inertial measurement units have been used to estimate vGRFs during walking. Veras et al. (2022) found that a hip-worn accelerometer narrowly outperformed other accelerometers (distal shank, lower back) in estimating vGRF loading peak during walking (hip: R2>0.96, mean absolute percentage error <7.3%). Similarly, Alcantara et al. (2021) estimated loading peak vGRFs during running using a sacral accelerometer within a mean absolute error (MAE) of 4.3% BW (percent body weight) using a quantile regression forest model and of 4.0% BW using a linear regression model. Even more researchers have used accelerometers below the waist to estimate vGRF. Jiang et al, (2020) found a shank accelerometer to be the single best estimator of vGRF (within an root mean square error of 2%) compared to other sensors on the foot, distal thigh, and proximal thigh. However, those authors did not include comparisons to a waist-worn or hip-worn sensor. Bach et al. (2022) used only shank accelerometers to estimate vGRF profiles with R2=0.97 and a normalized root mean square error of 5.2%. Altogether, these studies highlight that various accelerometer numbers and locations can be used to estimate vGRF. However, none of these studies actively prescribed vGRF changes to determine veracity in predicting effects of loading interventions or to drive those interventions directly.

Based on the available literature, researchers and clinicians seeking to estimate walking vGRF via accelerometry may be confused regarding where to place sensors and how many are needed for accurate outcomes. Thus, our first goal was to determine how many sensors and which locations yielded the most accurate vGRF loading peak estimates. Those individuals may also wonder whether wearable devices can reliably detect changes in loading profiles during walking. Accordingly, our second goal was to characterize how different loading conditions prescribed using biofeedback affect vGRF loading peak estimates. Ultimately, this is an important step in developing wearable sensor and biofeedback systems that can be deployed in real-world environments to detect, monitor, and treat aberrant forces associated with lower extremity osteoarthritis.

### Methods

We recruited a cohort of 20 (10 male, 10 female) healthy young adults to participate in this study (age: 24.7±5.18 years, height: 1.77±0.11 m, mass: 75.6±13.7 kg, typical walking speed: 1.41±0.09 m/s). We excluded any prospective participants who: were younger than 18 or older than 35 years of age; had a history of congenital or acquired cognitive, ophthalmologic, or neurological disorders; had a history of chronic peripheral or central vestibular disorders; began anti-seizure medication for any reason within two months of participation; had a BMI equal to or greater than 36; or used an assistive device and/or orthotics to walk. We measured participants' habitual overground walking speed via four passes along a 10-m walkway. We used Delsys Trigno (Natick, MA, USA) sensors to collect bilateral three-dimensional acceleration from the dorsum of each foot, the anterior/medial bony surface of the distal shank, and posteriorly on the waist.

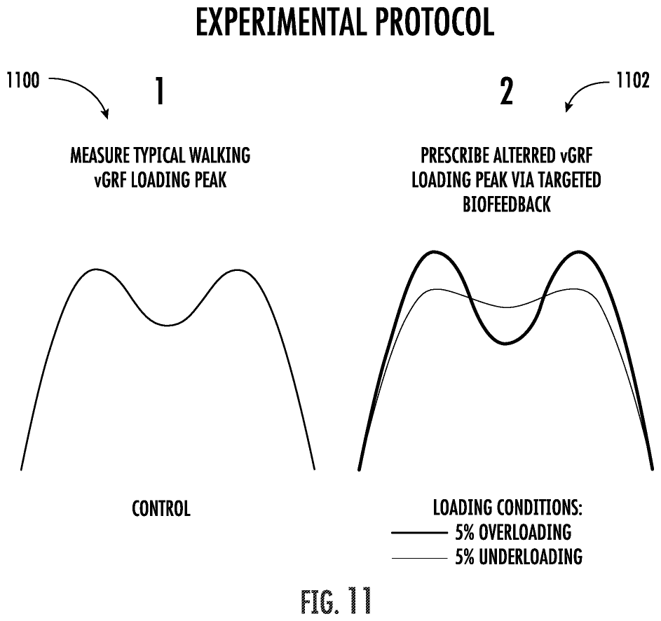

FIG. 11 shows a chart 1100 of vGRF measurements along a percent gait cycle in a control condition and a chart 1102 of vGRF measurements along a percent gait cycle in loading conditions, namely 5% overloaded and 5% underloaded. We recorded ground reaction forces and lower body accelerations during typical walking and prescribed changes using vGRF targeted biofeedback displaying the loading peak on a screen in front of the participant. Participants walked at their typical overground speed on a force-instrumented treadmill (Bertec, Columbus, OH, USA) for five minutes (control condition, FIG. 11). We recorded synchronized acceleration (2000 Hz) and GRF data (1200 Hz) via Qualisys Track Manager (Qualisys, Gothenburg, Sweden). During the third minute of this control condition, we also recorded vGRF profiles using custom Matlab (Mathworks, Natick, MA, USA) scripts to use in subsequent trials with targeted biofeedback, as shown in FIG. 12 with visual display 110 of visual biofeedback. Specifically, participants performed two additional five-minute walking trials while responding to real-time biofeedback designed to prescribe, in randomized order, 5% higher and 5% lower loading peak vGRF (overloading and underloading, respectively) during the first 50% of the stance phase (FIG. 11). Referring again to FIG. 12, participants viewed a screen showing two bar plots, representing their left and right vGRF loading peak, updated in real time as the average from the two previous steps in visual biofeedback 1202. The ±5% target values for these loading conditions were based on the side-specific averages measured during the control condition and were designed to emulate different lower extremity loading phenotypes. To facilitate synchronization, we asked participants to stomp on each force plate with their left and right foot in succession prior to starting or stopping the recorded data. The pre/post-trial stomp provided a sufficient timestamp via a high magnitude signal for a reliable cross correlation analysis.

### Data Reduction & Model Descriptions

Out of the 60 total trials recorded, two trials needed to be manually adjusted due to a timing error between the acceleration and force data. For these two trials, we synchronized the acceleration and force data by identifying the relative lag using a cross-correlation function and truncating or appending empty frames to the acceleration signal, keeping the force signal constant.

We extracted the first 50 valid left and right strides for a total of 100 strides per walking condition. We resampled acceleration and GRF signals to 100 Hz in post processing. For consistency across both vGRF and acceleration signals, we extracted gait cycles using a peak-finding algorithm on body weight (BW) normalized force data, identifying stance phases with a minimum height of 0.95 BW, a minimum prominence of 0.8 BW, a minimum width of 0.15 s, and a minimum distance of 0.5 s between peaks. We selected these identification parameters based on manual observation across the entire dataset (all subjects and biofeedback conditions).

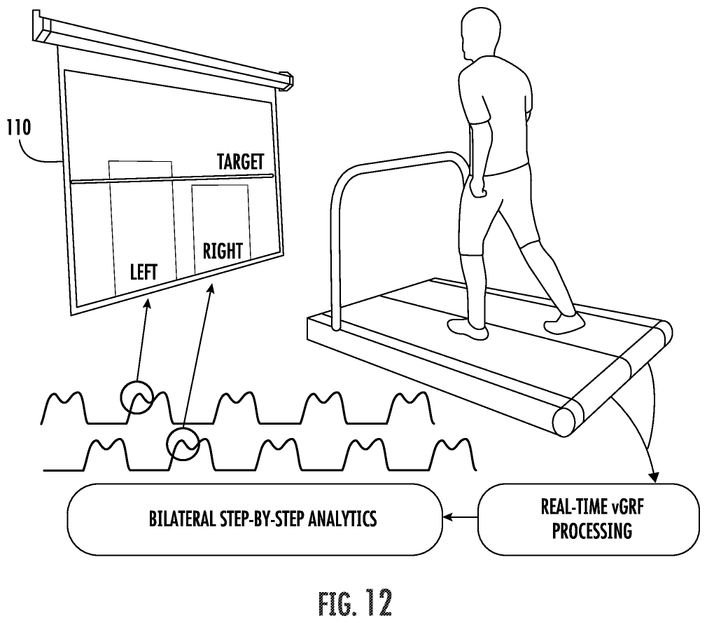

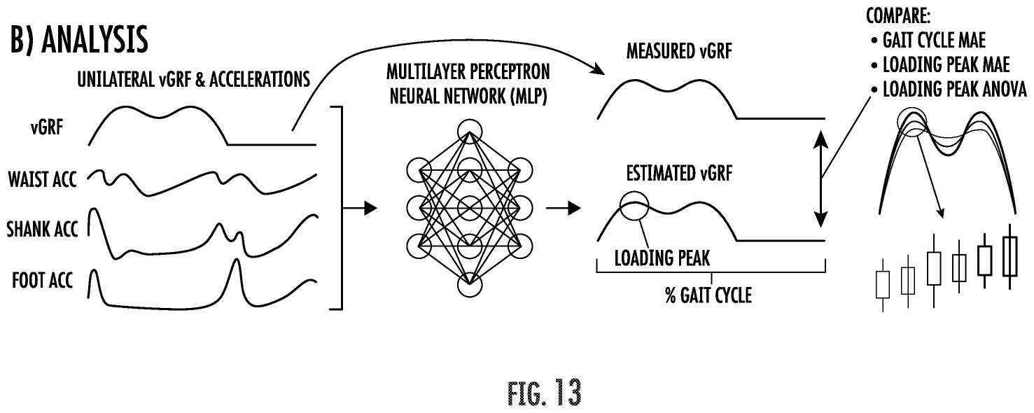

FIG. 13 shows a schematic diagram of the analysis steps. We extracted accelerometer signals in time series for foot, shank, and waist with time-matched vGRF data and resampled each stride to 100 data points. We calculated 3D acceleration vector magnitudes (via Euclidian normalization) to remove any bias of sensor orientation and sidedness. For analysis, we trained a series of MultiLayer Perceptron models to estimate the unilateral vGRF across the gait cycle based on acceleration inputs from various sensor combinations that included waist, shank, and foot sensors. All trained models unilaterally estimated vGRF, only including sensors on that specific side (i.e., left vGRF from the waist, left shank, and left foot).

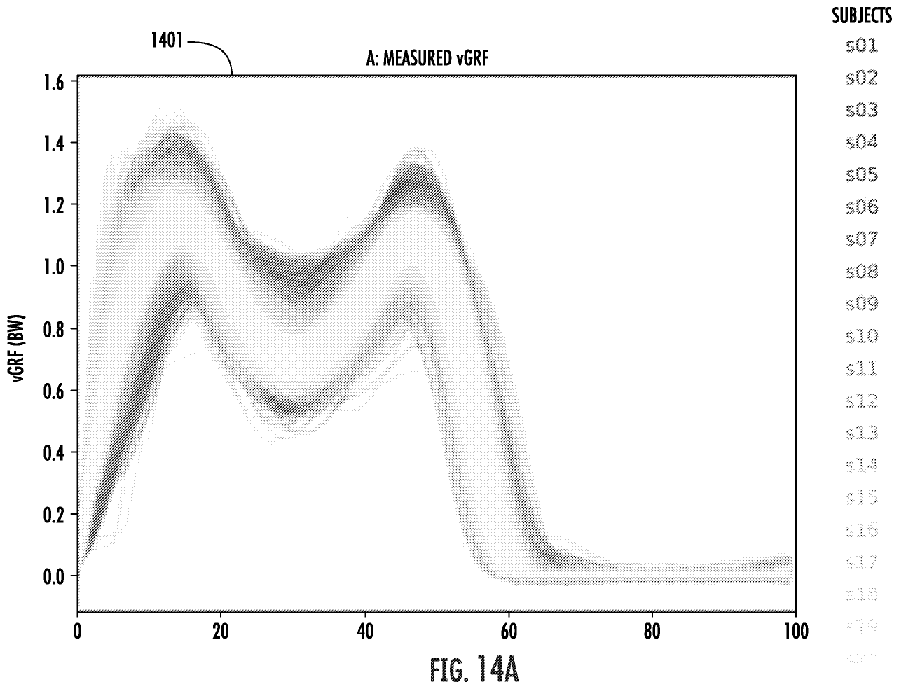

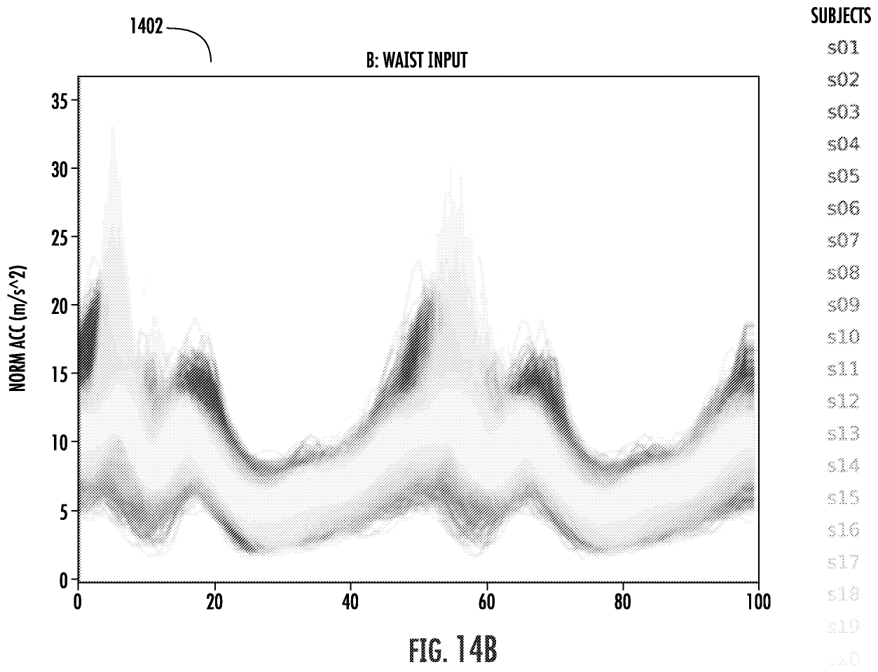

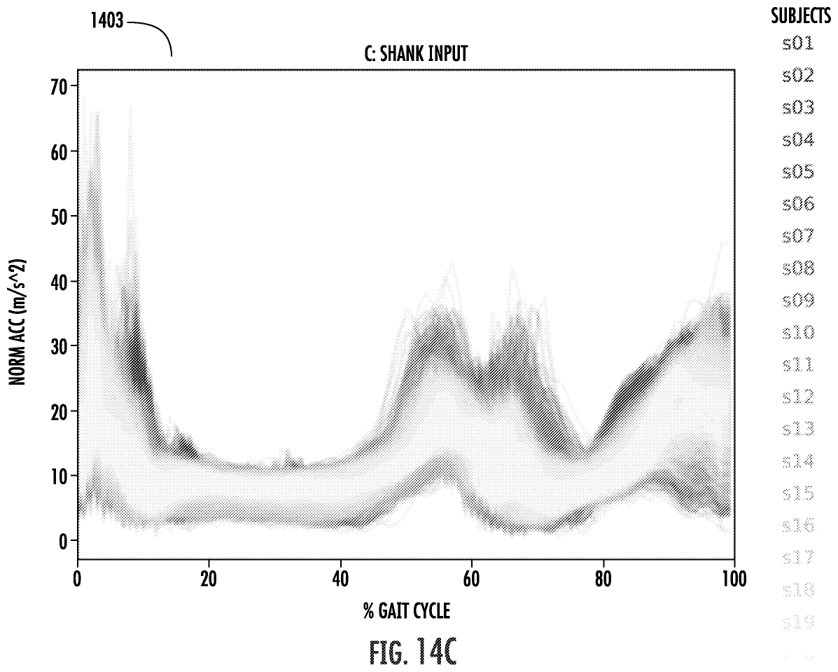

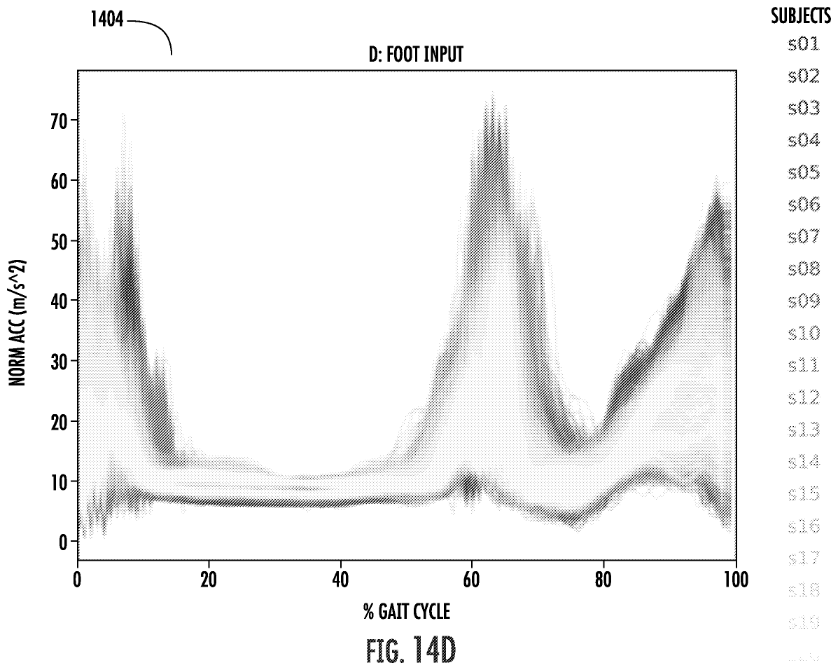

FIGS. 14A-14D show the measured vGRF and acceleration data for all input steps. FIG. 14A shows a graph 1401 of the target vGRF signal in relation to BW over a gait cycle. FIGS. 14B-14D show input vector magnitude acceleration waveforms used to estimate vGRF for 100 steps (50 left, 50 right) for all subjects (separated by color). FIG. 14B shows a graph 1402 of the measured normal acceleration at the waist over a gait cycle. FIG. 14C shows a graph 1403 of the measured normal acceleration at the shank over a gait cycle. FIG. 14D shows a graph 1404 of the measured normal acceleration at the foot over a gait cycle.

We tested seven different sensor combinations by altering the number and location of accelerometers used as model inputs: foot, shank, and waist (FSW); foot and waist (FW); shank and waist (SW); foot and shank (FS); shank only (S); foot only (F); and waist only (W). Five Delsys Trigno Accelerometers were used in total: one for the waist, one for each shank, and one for each foot. Each model included steps across all loading conditions (control/under/over). We trained Multi-Layer Perceptron regressor models from the Sci-Kit Learn, with a convergence max iteration of 500, logistic activation functions, and a single hidden layer (size=200 neurons) based on preliminary parameter tuning. To benchmark model accuracies, we performed 5-fold cross validation, splitting the training and testing steps on an 80:20% (16:4 subjects) ratio in a subject leave-out approach. In line with common k-fold cross validation practices, we changed which 20% the model was tested on for each k-fold iteration.

### Statistical Comparisons

We quantified accuracy via mean absolute error (MAE) between measured and predicted vGRF both across the entire gait cycle (GC) and at the loading peak. We characterized the ability to distinguish between loading conditions by performing two-way repeated measures ANOVA using each of the three most-accurate models, testing for main effects of mode (measured vs estimated), condition (under, control, & over-loading), and for interaction effects (mode×condition). We also ran one-way repeated measures ANOVA across the MAE estimates across the GC and at the loading peak. When a significant main effect was found, Tukey's post-hoc tests identified significant pairwise differences between measurement modes and across loading conditions. We report effect sizes for ANOVAs as partial eta squared (ηp2) and post-hoc analyses using eta squared (η2).

### Results

### Benchmarking Accuracy Across Sensor Configurations

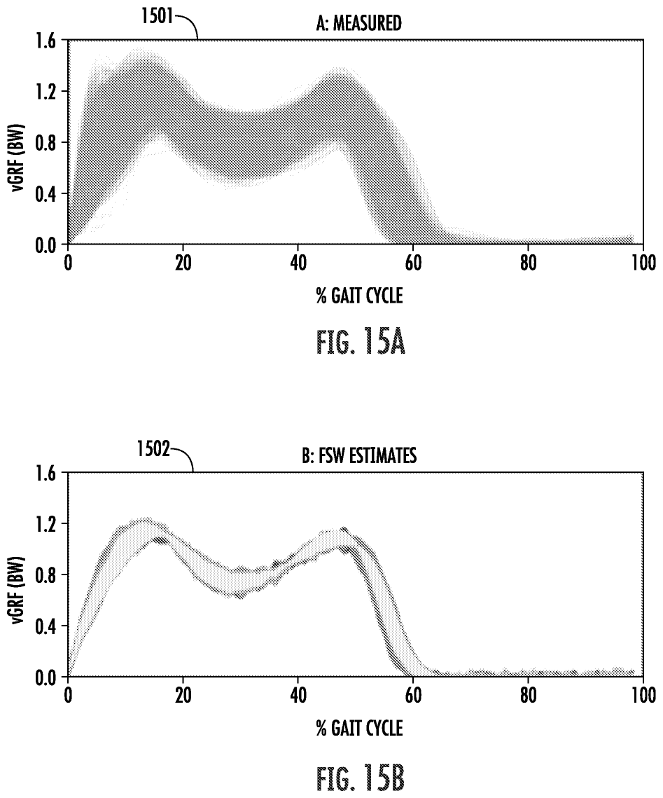

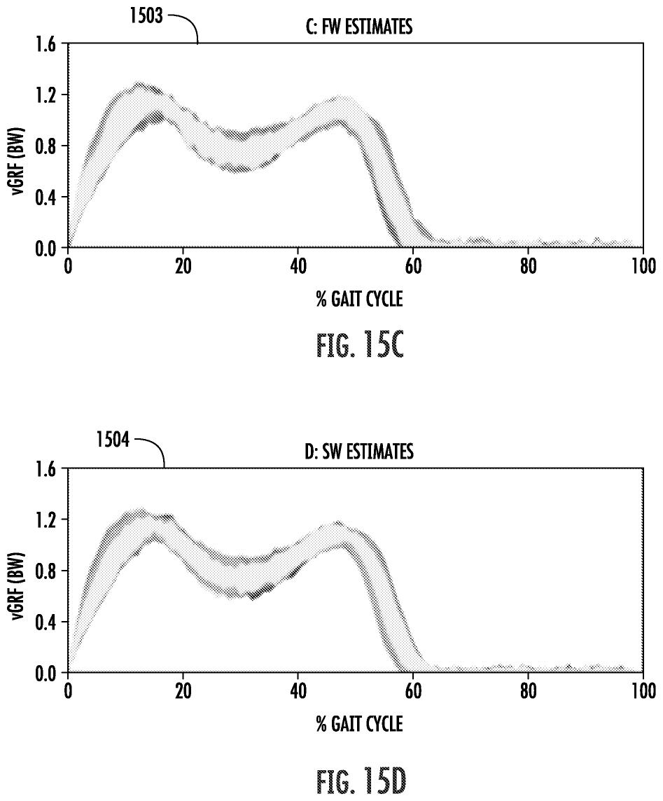

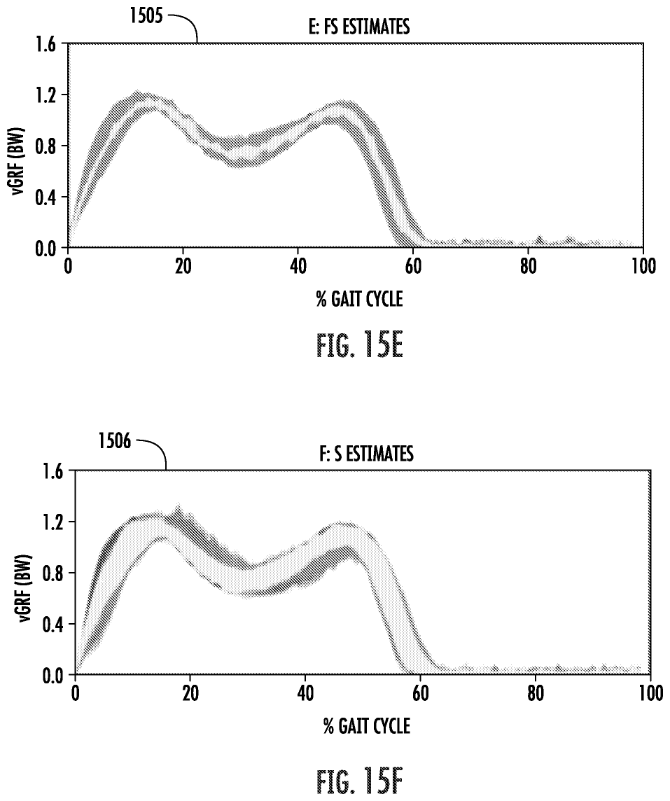

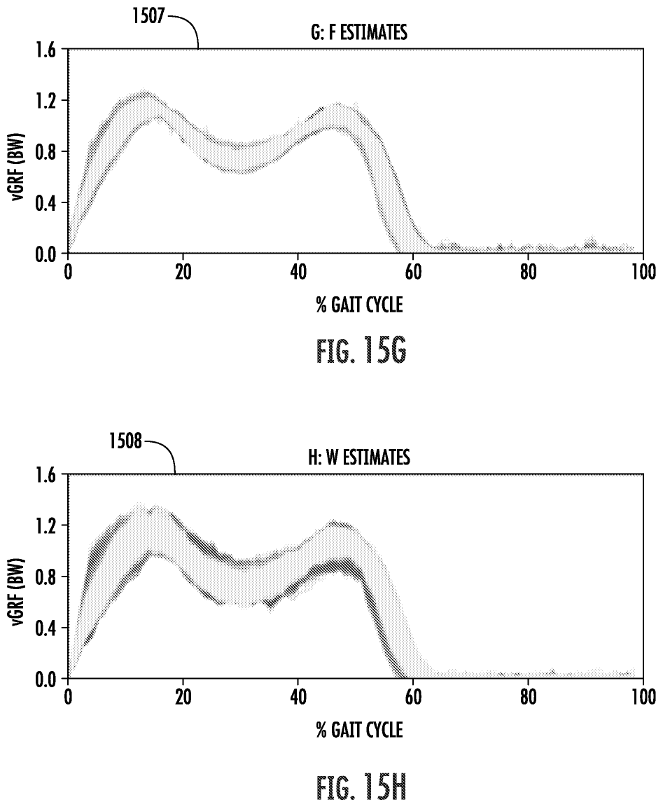

FIGS. 15A-15H show all measured and predicted vGRFs for all steps in the testing set across each configuration (panels) and k-fold iteration (colors). FIG. 15A shows a graph 1501 of the measured vGRF signal in relation to BW over a gait cycle. FIG. 15B shows a graph 1502 of FSW estimates of vGRF in relation to BW over a gait cycle. FIG. 15C shows a graph 1503 of FW estimates of vGRF in relation to BW over a gait cycle. FIG. 15D shows a graph 1504 of SW estimates of vGRF in relation to BW over a gait cycle. FIG. 15E shows a graph 1505 of FS estimates of vGRF in relation to BW over a gait cycle. FIG. 15F shows a graph 1506 of S estimates of vGRF in relation to BW over a gait cycle. FIG. 15G shows a graph 1507 of F estimates of vGRF in relation to BW over a gait cycle. FIG. 15H shows a graph 1508 of W estimates of vGRF in relation to BW over a gait cycle. Qualitatively, all sensor configurations yielded model predictions that reflected the double-hump shape of vGRF across the gait cycle. Compared to other models, the Waist model (FIG. 15H) appeared to best reflect the variability of measured vGRFs across participants and conditions (FIG. 15A).

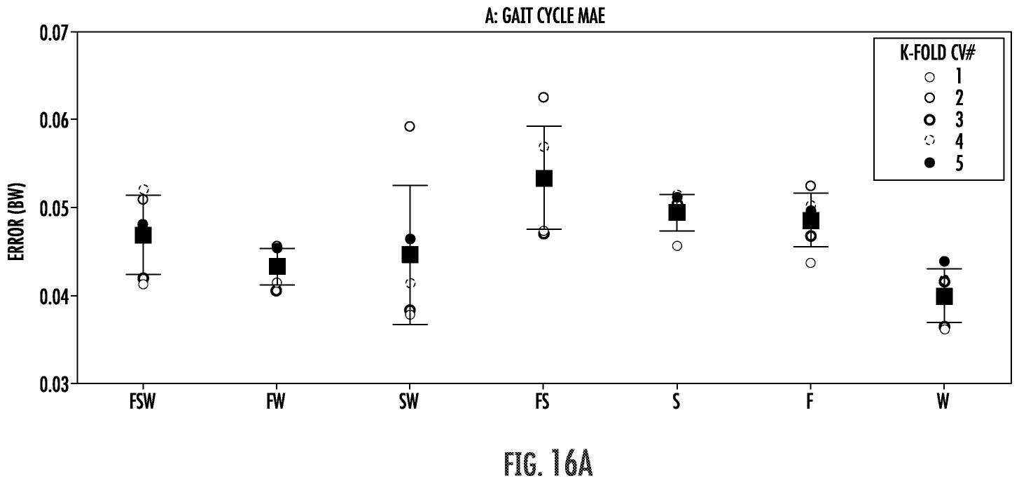

FIGS. 16A and 16B show MAE between measured and estimated vGRFs over the gait cycle and at the instant of the loading peak, respectively, for each sensor configuration (x-axis) and k-fold iteration (colors). Model accuracy varied across each of the sensor combinations yet was relatively stable across each k-fold iteration. The model driven exclusively by Waist accelerations outperformed all other models (GC MA: 4.0% BW, loading peak MAE: 4.3% BW). The Foot-Waist configuration performed second best (GC MAE: 4.3% BW, loading peak MAE: 4.9% BW) followed by the Shank-Waist configuration (GC MAE: 4.5% BW, loading peak MAE: 5.2% BW). All other sensor configurations produced estimates with higher than these reported MAE values, and generally above 5% BW.

### Model-Predictions of Altered Lower-Extremity Loading

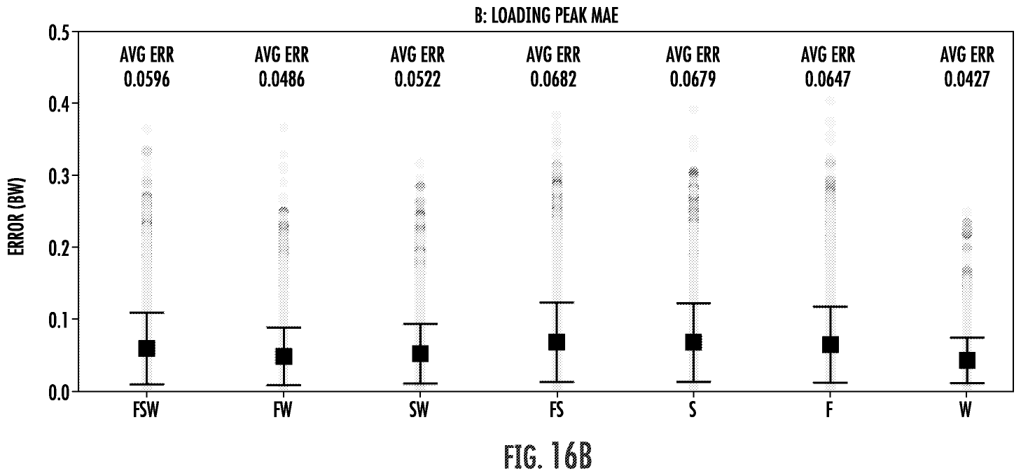

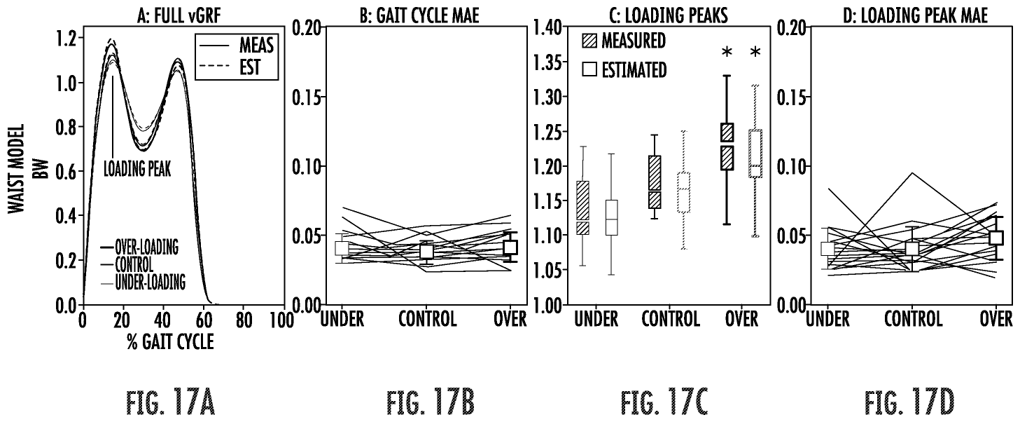

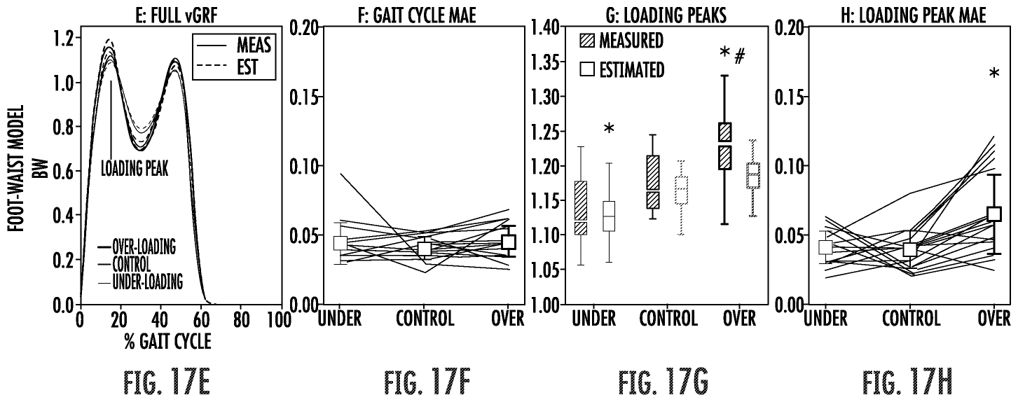

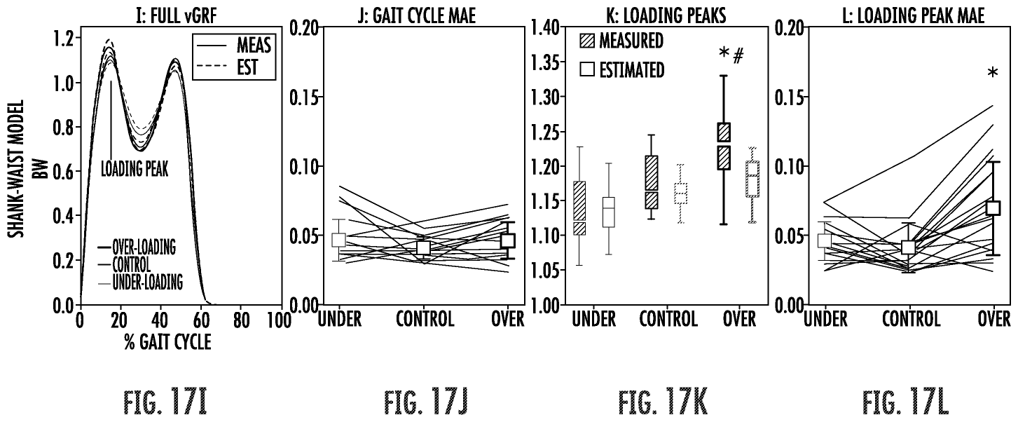

FIGS. 17A-17D show results from the Waist model, FIGS. 17E-17H show results from the Foot-Waist model, and FIGS. 17I-17L show results from the Shank-Waist model across each loading condition (underloading, control, and overloading). Asterisks (*) indicate a significant difference from the control condition for that measurement type, whereas hashes (#) represent a significant difference between measurement modalities (true vs estimated). FIGS. 17A, 17E, and 17I show graphs of measured vGRFs for the Waist model, Foot-Waist model, and Shank-Waist model, respectively. FIGS. 17B, 17F, and 17J show graphs of gait cycle MAEs for the Waist model, Foot-Waist model, and Shank-Waist model, respectively. FIGS. 17C, 17G, and 17K show graphs of loading peaks for the Waist model, Foot-Waist model, and Shank-Waist model, respectively. FIGS. 17D, 17H, and 17L show graphs of loading peak MAEs for the Waist model, Foot-Waist model, and Shank-Waist model, respectively. Compared to the control condition, the measured and average estimated vGRF loading peaks increased and decreased consistently with the biofeedback target values (condition main effect: p<0.001, ηp2=0.719). Compared to the control condition, the measured vGRF loading peaks significantly differed only for the overloading condition (Tukey's: p=0.009, η2=0.177, FIGS. 17C, 17G, 17K).

The Waist model successfully distinguished between overloading and control vGRF loading peaks (condition: p<0.001, ηp2=0.719) and yielded values that were indistinguishable from those measured (mode: p=0.160, ηp2=0.101, FIG. 17C). Waist model MAEs did not significantly change between conditions across the gait cycle (condition: p=0.368, ηp2=0.051, FIG. 5B) nor at the loading peak (condition: p=0.222, ηp2=0.076, FIG. 17D).

Compared to the control condition, the Foot-Waist model (condition: p<0.001, ηp2=0.722) did not distinguish loading peaks for overloading (p=0.092, η2=0.107), but did yield a significant difference for underloading (p=0.026, η2=0.149, FIG. 17G). Compared to measured values, the Foot-Waist model yielded a different loading peak vGRF for overloading (p=0.023, η2=0.124, FIG. 17G) but not underloading (p>0.533, η2<0.010). The Foot-Waist model also yielded similar estimates of the vGRF across the gait cycle (condition: p=0.386, ηp2=0.049, FIG. 17F). Conversely, Foot-Waist model accuracy at the loading peak varied across conditions (condition: p<0.001, ηp2=0.362, FIG. 17H) with overloading error significantly higher than for underloading and control conditions (p≤0.001, η2=0.224).

The Shank-Waist model (condition: p<0.001, ηp2=0.708) was unable to distinguish between loading conditions (p>0.064, η2=0.138, FIG. 17K). Additionally, the Shank-Waist model estimates were different from the measured vGRF loading peaks (mode: p=0.025, η2=0.239), with the overloading condition estimates different from measured values (p=0.009, η2=0.161, FIG. 17K). The Shank-Waist model yielded estimates with consistent errors between conditions across the gait cycle (condition: p=0.205, ηp2=0.080, FIG. 17J). However, the Shank-Waist model errors at the loading peak varied between conditions (condition: p<0.001, ηp2=0.411, FIG. 17L) with overloading error significantly higher than underloading and control conditions (p≤0.002, η2≥0.163).

### Seeking Interpretability

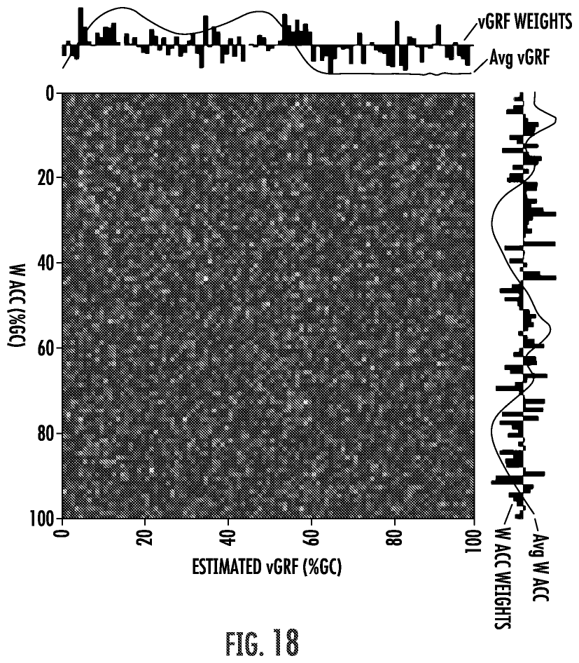

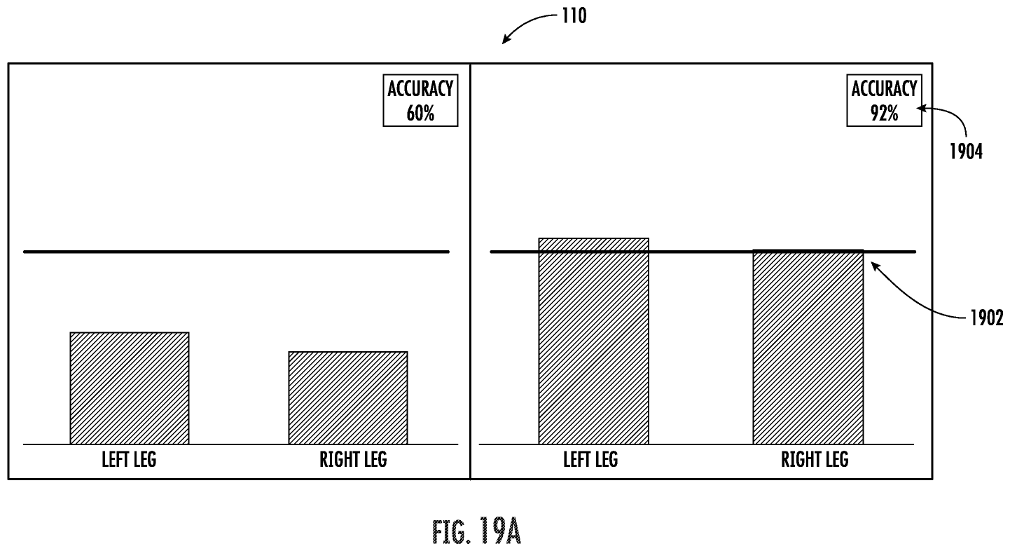

FIG. 18 is graph comparing weighted acceleration and estimated vGRF. FIG. 18 attempts to explore characteristics of the neural-network prediction model for the Waist-only sensor configuration. To address interpretability of the Waist model, we show the resultant model weights for the input (vertical axis) and output (horizontal axis) layers of our multilayer perceptron neural network model. Model weights are shown via heatmap across their respective time series (along each axis top to bottom and left to right), with higher positive weights in red, lower negative weights in blue, and zero weights in black. Along each sub-axis, we also show the mean-normalized summed weights for each column/row as a bar graph, with higher relative weights shown above or to the right of the axes. For additional context, we also overlay the average input Waist vector magnitude acceleration signal along the vertical sub-axis and the average estimated vGRF signal along the horizontal sub-axis, both shown in gray. The estimated vGRF (horizontal axis and top sub-axis) received strong positive weights during early (5-15% GC) and late (50-60% GC) stance phase, as well as strong negative weights during leg swing (60-100% GC). Although the vGRF weights during midstance (15-50% GC) included both positive and negative values, those weights were relatively smaller. Model weights for the input Waist acceleration vectors (vertical axis and right sub-axis) were more complicated. However, we tended to see more positive weights during the stance phase (0-50% GC) and more negative weights during the swing phase (60-100% GC).

### DISCUSSION

Our goals were to: 1) determine how many sensors and what sensor placement and locations yielded the most accurate vGRF loading peak estimates during walking; and 2) characterize how prescribing different loading conditions affected vGRF loading peak estimates. We found that a neural network using one waist-mounted accelerometer yielded the most accurate loading peak vGRF estimates during walking, with errors around 4.3% BW. Furthermore, this waist-only configuration was able to distinguish between control and overloading conditions prescribed using biofeedback, matching measured vGRF outcomes. Including foot or shank acceleration signals in the model reduced accuracy, particularly for the overloading condition. Our results suggest that a system designed to monitor changes in walking vGRF or to deploy targeted biofeedback may only need a single accelerometer located at the waist.

The novelty in our scientific contributions is twofold. First, we systematically compared model-prediction accuracies across multiple sensor configurations. Previous studies designed to predict walking vGRF values from wearable sensors have generally quantified model accuracies based on a single accelerometer placed at, for example, the feet versus shank or waist. By using multiple combinations of sensors, we see the effects of how the various sensor locations affect vGRF estimates (FIGS. 14A-14D). A second innovation comes from our characterization of accelerometers to detect changes in vGRF across multiple loading phenotypes, prescribed herein using real-time biofeedback. As one clinical application, vGRF profiles during walking are indicative of patient-reported and biological outcomes among individuals during recovery from anterior cruciate ligament (ACL) reconstruction. Thus, this study suggests that wearable sensors can be used to detect and monitor vGRF changes between overloading and typical walking, which could help improve clinical outcomes during rehabilitation.

Generally, our measured vGRFs were able to differentiate between control and overloading conditions and the Waist model replicated those findings. Interestingly, the Foot-Waist model detected a significant difference between underloading peak vGRF and the control condition that was not detected using the measured vGRF signals (FIG. 17G). Although loading peaks did decrease on average during the underloading condition, no other model or measurement mode detected a significant difference from the control vGRF. Loading peak errors were also higher on average during the underloading condition (FIG. 17H), which may have contributed to this unanticipated finding. In combination with inaccuracy during overloading, we suggest that the Foot-Waist configuration is unlikely to yield accurate vGRF loading peak estimates across biofeedback conditions.

Our Waist model aligns with other groups that have used machine learning or regression equations to estimate vGRF loading peak during walking or running. Although lower leg sensor sites can accurately estimate vGRF signals under different scenarios, we found that accelerometers placed near the body's center of mass may provide optimal features to estimate vGRF during walking. This outcome is theoretically supported by simple Newtonian physics-based models of walking, in that acceleration of the body's center of mass is proportional to the net external force acting on the body. While the other lower-limb sensors may contain salient features that can accurately estimate vGRF, we contend that sensor locations that track center of mass acceleration are more reliable and accurate.

We chose an accelerometer-based solution for vGRF estimation because of their low cost and simplicity, both of which are beneficial to delivering options for estimating vGRF outside of the research laboratory. Compared to other potential solutions (pressure-sensing insoles, instrumented walkways), accelerometers and inertial measurement units are adaptable across patients of any size, simple to change between patients, and can be applied to a wide variety of outpatient environments. Unlike previous studies, we used the acceleration vector magnitude in our model, combining the 3-dimensional components into one composite acceleration measure. This processing strategy simplifies sensor placement orientation, is agnostic to left/right sidedness, and reduces data processing complexity which should enable high-throughput assessment.

As scientists, engineers, and clinicians increasingly utilize machine learning models to estimate biomechanical parameters, interpretation of the data produced by these models remains a challenge for successful utilization of model-based techniques. We assessed the interpretability of our most accurate model by mapping the input and output weights from the neural network multilayer perceptron model (FIG. 18). We found that model weights tended to align with notable events from the gait cycle, including stance and swing phase as well as local maximums in the vGRF signal during early and late stance. Our interpretability plot reveals some of the underlying features that our model may use to estimate the vGRF waveform. By calculating and displaying our model weights, we aim to work toward a better understanding of how machine learning models make predictions, and what input features are most influential, with the hope of understanding and explaining model estimates.

### CONCLUSION

We can accurately estimate the vGRF loading peak within 5% of body weight and distinguish between biofeedback-induced loading conditions using only a waist accelerometer by implementing a multilayer perceptron neural network model. However, other lower-body segment accelerations may be helpful in other ways, identifying gait events which are necessary to parse gait cycles from the acceleration signal stream. We suggest that a wearable sensor system comprised of a single waist sensor is not only sufficient, but optimal for implementing a biofeedback-based therapy system to prescribe limb loading during gait retraining.

FIGS. 19A-19C show examples of visual displays 110 that include visual biofeedback. The visual biofeedback can include a visual representation of the real-time generated vGRF biofeedback prediction data for the right foot and left foot a visual representation of an optimal vGRF threshold level 1902 for the subject to compare with the prediction data. Visual display 110 can includes an accuracy reading 1904 with a numerical value based on the proximity between the real-time vGRF of either foot and the vGRF threshold level. Visual display 110 can provide a visual indicator that identifies whether the subject is on target, as shown in FIG. 19B, or deviating from the target, as shown in FIG. 19C. Visual display 110 can be presented on the central host computing device.

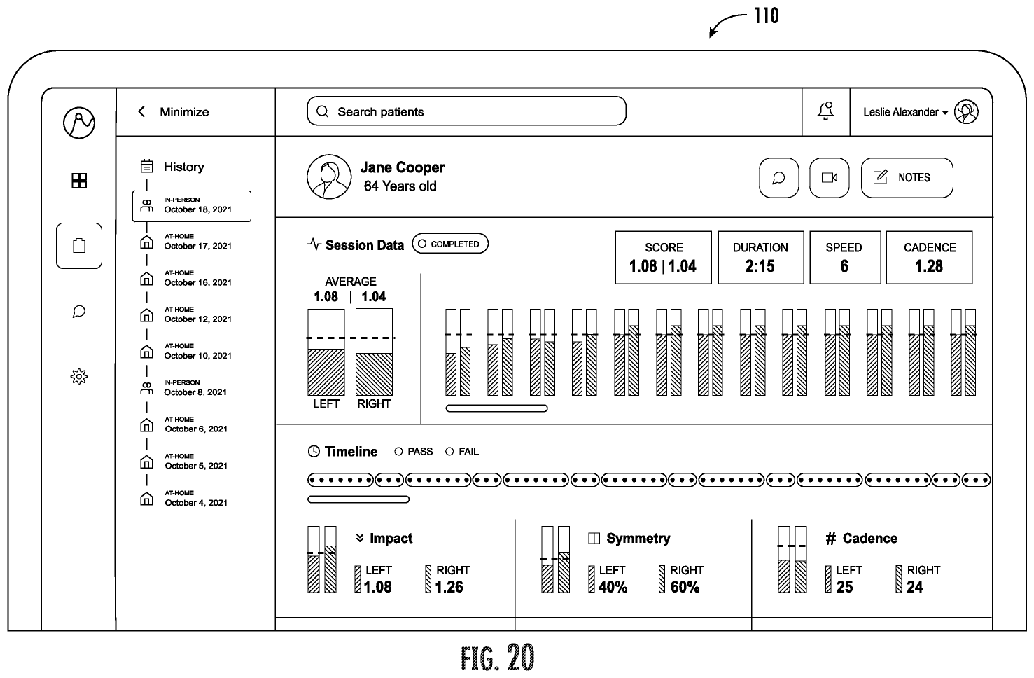

FIG. 20 shows an example visual display 110 summarizing a walking session by a subject on a personal computer. Visual display 110 can include a personalized profile for the subject with various statistics including average and/or real-time speed and cadence. Visual display 110 can include a duration for the walking session, an average and/or real-time display of vGRFs for each foot and a score based on the proximity of the average vGRFs to the vGRF threshold level.

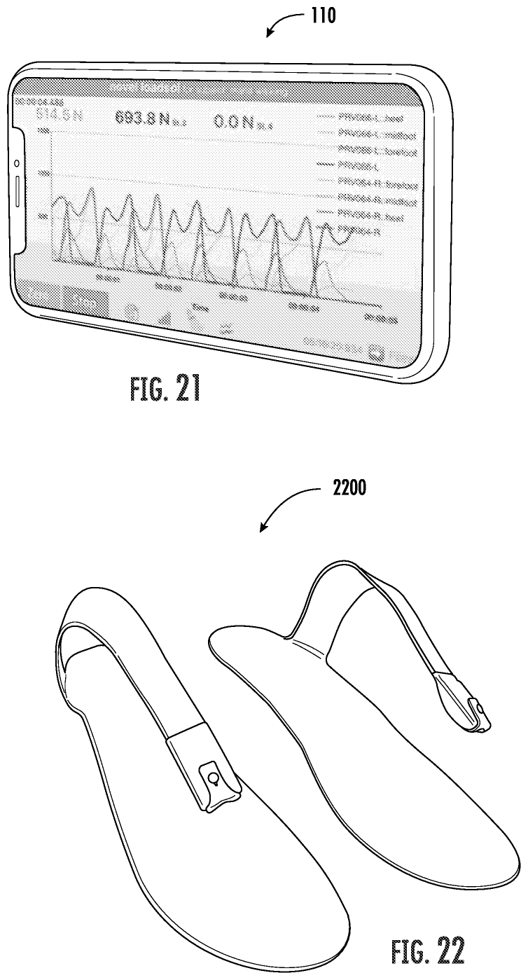

FIG. 21 shows another example visual display 110 on a personal computer. In this example, visual display 110 includes a graph of the generated vGRFs over time. The personal computer can include the central host computing device.

FIG. 22 shows example footfall pressure sensors 2200. Footfall pressure sensor 2200 is worn on the bottom of a subject's foot and is configured to measure a pressure on the bottom of the foot. As described herein, footfall pressure sensors 2200 can be used in conjunction with other wearable sensor devices 102 placed in various locations on a subject's body. Wearable sensor device 102 may include an ADC input to connect to footfall pressure sensors 2200.

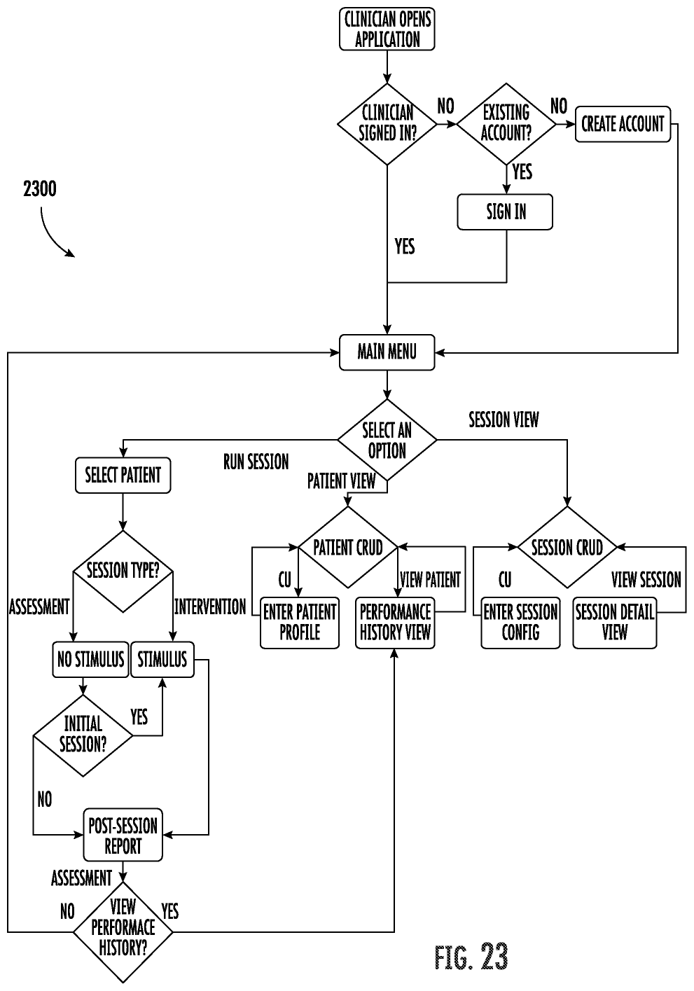

FIG. 23 is a flow chart 2300 illustrating an example clinician workflow on a user interface for the subject matter described herein. The clinician workflow includes creating an account on a user interface if the clinician does not already have an account, which allows the clinician to then select a patient to view the patient's profile or commence a walking session for the patient, from which data will be collected for the patient's profile.

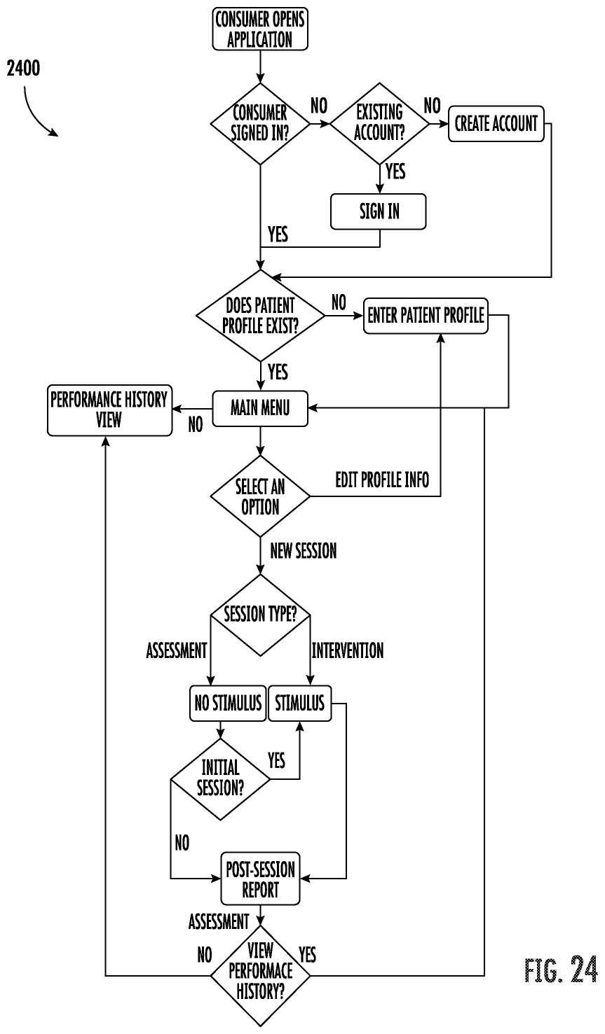

FIG. 24 is a flow chart 2400 illustrating an example consumer workflow on a user interface for the subject matter described herein. The user interface can be configured for a consumer to use outside of a doctor or physical therapist's office and without supervision. The consumer can create or login to a user account and view a profile including information from previous walking sessions. The consumer can commence a walking session and may select a desired duration. The user interface can provide a report of a completed walking session including average vGRFs and overall accuracy compared to an optimal vGRF threshold level. The user interface may provide real-time biofeedback of predicted vGRFs for the consumer to see during a walking session.

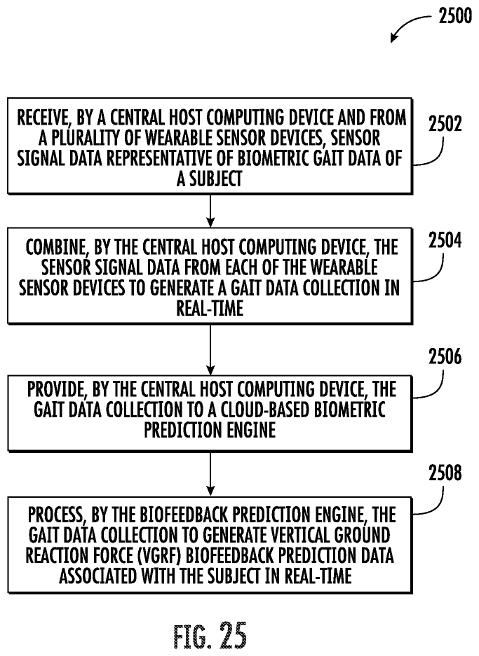

FIG. 25 is a flow chart illustrating an example method for detecting and monitoring gait using a portable gait biofeedback system. In some embodiments, method 2500 depicted in FIG. 25 is an algorithm, program, or script stored in memory that when executed by a processor performs the steps recited in blocks 2502-2508. In some aspects, method 2500 represents a list of steps embodied as software code and/or logic of a host computing device.

In block 2502, method 2500 includes receiving sensor signal data representative of biometric gait data of a subject by a central host computing device and from a plurality of wearable sensor devices. A central host computing device can be configured to receive the sensor signal data from a plurality of wearable sensor devices. In alternate aspects, a base station unit (or central hub transceiver device) can be adapted to receive the sensor signal data from the plurality of wearable sensor devices before forwarding the data to the central host computing device. The wearable sensor devices can include one wearable sensor device configured to be mounted on a waist of the subject and two wearable sensor devices configured to be mounted on either ankle of the subject. The wearable sensor device configured to be mounted on the waist can include an accelerometer. Wearable sensor devices can include wearable sensor devices configured to be mounted on thighs or feet of the subject. The plurality of wearable sensor devices can include at least one of: a motion sensor device, a force measurement sensor device, an accelerometer device, an inertial measurement unit (IMU) device, or a plantar pressure sensor device.

In block 2504, method 2500 includes combining, by the central host computing device, the sensor signal data from each of the wearable sensor devices to generate a gait data collection in real-time. The central host computing device can be configured to request (e.g., ping) each of the sensor devices to send the sensor signal data to the central host computing device. In alternate aspects, this functionality may be performed by the base station unit. After receiving the sensor signal data, the central host computing device may be adapted to combine/accumulate the signal data and generate a gait data collection or package. The biofeedback management engine can be responsible for pinging the sensor devices and/or generating the collection of gait data. The gait collection data can include at least one of motion data or bilateral pressure data.

In block 2506, method 2500 includes providing, by the central host computing device, the gait data collection to a cloud-based biometric prediction engine. The central host computing device and/or biofeedback management engine can be configured to send the gait data collection to the biometric prediction engine supported by a cloud-based host or infrastructure via a network (e.g., the Internet).

In block 2508, method 2500 includes processing, by the biofeedback prediction engine, the gait data collection to generate vertical ground reaction force (vGRF) biofeedback prediction data associated with the subject in real-time. The biofeedback prediction engine can produce vGRF biofeedback prediction data associated with the patient or user using the received gait data collection in real-time. The biofeedback prediction engine can be further configured to transmit the generated vGRF biofeedback prediction data back to the central host computing device for presentation (e.g., via visual display) to the subject (e.g., patient and/or physician). The biofeedback prediction engine can be executed by a machine learning algorithm. The vGRF biofeedback prediction data can be provided to the central host computing device for visual display on a user interface in real-time. The vGRF biofeedback prediction data can include a visual representation of one or more threshold levels of ground reaction force that compels the subject to alter step-by-step gait biomechanics in real-time. The vGRF biofeedback prediction data can be provided to the central host computing device for auditory notifications on a user interface in real-time. Auditory notifications can include notifications of current vGRF predictions for the right and left foot and/or a comparison between the current vGRF predictions and threshold levels for vGRF, for example accuracy percentages.

It will be understood that various details of the presently disclosed subject matter may be changed without departing from the scope of the presently disclosed subject matter. Furthermore, the foregoing description is for the purpose of illustration only, and not for the purpose of limitation.

The disclosure of each of the following references is hereby incorporated herein by reference in its entirety.

### REFERENCES

1. S. L. Chaparro-Cárdenas, A. A. Lozano-Guzmán, J. A. Ramirez-Bautista, and A. Hernández-Zavala, “A review in gait rehabilitation devices and applied control techniques,” Disability and Rehabilitation: Assistive Technology, vol. 13, no. 8, pp. 819-834, 2018/11/17 2018.
2. J. Kim, S. Jung, and C. Song, “The Effects of Auditory Feedback Gait Training Using Smart Insole on Stroke Patients,” Brain Sciences, vol. 11, no. 11, p. 1377, 2021.
3. T. Mikolajczyk et al., “Advanced technology for gait rehabilitation: An overview,” Advances in Mechanical Engineering, vol. 10, no. 7, p. 1687814018783627, 2018.
4. A. Evans-Pickett et al., “Biomechanical effects of manipulating peak vertical ground reaction force throughout gait in individuals 6-12 months after anterior cruciate ligament reconstruction,” (in eng), Clin Biomech (Bristol, Avon), vol. 76, p. 105014, June 2020.
5. B. A. Luc-Harkey, J. R. Franz, J. T. Blackburn, D. A. Padua, A. C. Hackney, and B. Pietrosimone, “Real-time biofeedback can increase and decrease vertical ground reaction force, knee flexion excursion, and knee extension moment during walking in individuals with anterior cruciate ligament reconstruction,” (in eng), J Biomech, vol. 76, pp. 94-102, Jul. 25 2018.
6. P. DEVITA and W. A. SKELLY, “Effect of landing stiffness on joint kinetics and energetics in the lower extremity,” Medicine & Science in Sports & Exercise, vol. 24, no. 1, pp. 108-115, 1992.
7. P. R. Cavanagh and M. A. Lafortune, “Ground reaction forces in distance running,” Journal of Biomechanics, vol. 13, no. 5, pp. 397-406, 1980/01/01/1980.
8. W. B. Edwards, T. R. Derrick, and J. Hamill, “Musculoskeletal Attenuation of Impact Shock in Response to Knee Angle Manipulation,” (in English), Journal of Applied Biomechanics, vol. 28, no. 5, pp. 502-510, 01 Nov. 20122012.
9. T. F. Novacheck, “The biomechanics of running,” (in eng), Gait Posture, vol. 7, no. 1, pp. 77-95, Jan. 1 1998.
10. K. Hasan, K. Biswas, K. Ahmed, N. S. Nafi, and M. S. Islam, “A comprehensive review of wireless body area network,” Journal of Network and Computer Applications, vol. 143, pp. 178-198, 2019/10/01/2019.
11. B. Pietrosimone et al., “Biochemical markers of cartilage metabolism are associated with walking biomechanics 6 months following anterior cruciate ligament reconstruction,” (in eng), J Orthop Res, vol. 35, no. 10, pp. 2288-2297, October 2017.
12. S. J. Pfeiffer et al., “Gait Mechanics and T1p MRI of Tibiofemoral Cartilage 6 Months after ACL Reconstruction,” (in eng), Med Sci Sports Exerc, vol. 51, no. 4, pp. 630-639, April 2019.
13. Pietrosimone, B. et al. Am. J. Sports Med. 44, 425-432 (2016).
14. Wellsandt, E. et al. Decreased Knee Joint Loading Associated With Early Knee Osteoarthritis After Anterior Cruciate Ligament Injury. Am. J. Sports Med. 44, 143-151 (2016).
15. Astephen, J. L., Deluzio, K. J., Caldwell, G. E., Dunbar, M. J. & Hubley-Kozey, C. L. Gait and neuromuscular pattern changes are associated with differences in knee osteoarthritis severity levels. J. Biomech. 41, 868-876 (2008).
16. Kumar, D. et al. Frontal plane knee mechanics and medial cartilage MR relaxation times in individuals with ACL reconstruction: A pilot study. The Knee 21, 881-885 (2014).
17. Kumar, D. et al. Frontal Plane Knee Mechanics and Early Cartilage Degeneration in People With Anterior Cruciate Ligament Reconstruction: A Longitudinal Study. Am. J. Sports Med. 46, 378-387 (2018).
18. Palmieri-Smith, R. M. & Thomas, A. C. A Neuromuscular Mechanism of Posttraumatic Osteoarthritis Associated with ACL Injury. Exerc. Sport Sci. Rev. 37, 147 (2009).
19. Pfeiffer, S. J. et al. Gait Mechanics and T1p MRI of Tibiofemoral Cartilage 6 Months after ACL Reconstruction. Med. Sci. Sports Exerc. 51, 630-639(2019).
20. Pietrosimone, B. et al. Biochemical markers of cartilage metabolism are associated with walking biomechanics 6-months following anterior cruciate ligament reconstruction. J. Orthop. Res. 35, 2288-2297 (2017).
21. Losina, E. et al. Lifetime Risk and Age at Diagnosis of Symptomatic Knee Osteoarthritis in the US. Arthritis Care Res. 65, 703-711 (2013).
22. Felson, D. T. Osteoarthritis as a disease of mechanics. Osteoarthritis Cartilage 21, 10-15 (2013).
23. Du, G. et al. Abnormal Mechanical Loading Induces Cartilage Degeneration by Accelerating Meniscus Hypertrophy and Mineralization After ACL Injuries In Vivo. Am. J. Sports Med. 44, 652-663 (2016).
24. Radin, E. L. et al. Effects of mechanical loading on the tissues of the rabbit knee. J. Orthop. Res. 2, 221-234 (1984).
25. Andriacchi, T. P., Koo, S. & Scanlan, S. F. Gait Mechanics Influence Healthy Cartilage Morphology and Osteoarthritis of the Knee. J. Bone Joint Surg. Am. 91, 95-101 (2009).
26. Crook, B. S. et al. Effect of walking on in vivo tibiofemoral cartilage strain in ACL-deficient versus intact knees. J. Biomech. 116, 110210 (2021).
27. Torry, M. R., Decker, M. J., Viola, R. W., O'Connor, D. D. & Richard Steadman, J. Intra-articular knee joint effusion induces quadriceps avoidance gait patterns11Presented at the XVII Congress of the International Society of Biomechanics, Calgares, Canada, 8-13 Aug. 1999. Clin. Biomech. 15, 147-159 (2000).
28. Pietrosimone, B. et al. Changes in voluntary quadriceps activation predict changes in muscle strength and gait biomechanics following knee joint effusion. Clin. Biomech. 29, 923-929 (2014).
29. Seeley, M. K., Park, J., King, D. & Hopkins, J. T. A Novel Experimental Knee-Pain Model Affects Perceived Pain and Movement Biomechanics. J. Athl. Train. 48, 337-345 (2013).
30. Pietrosimone, B. et al. Walking Ground Reaction Force Post-ACL Reconstruction: Analysis of Time and Symptoms. Med. Sci. Sports Exerc. 51, 246-254 (2019).
31. Luc-Harkey, B. A. et al. Lesser lower extremity mechanical loading associates with a greater increase in serum cartilage oligomeric matrix protein following walking in individuals with anterior cruciate ligament reconstruction. Clin. Biomech. 60, 13-19 (2018).
32. Pietrosimone, B. et al. Walking gait asymmetries 6 months following anterior cruciate ligament reconstruction predict 12-month patient-reported outcomes. J. Orthop. Res. 36, 2932-2940 (2018).
33. Pietrosimone, B. et al. Greater Mechanical Loading During Walking Is Associated With Less Collagen Turnover in Individuals With Anterior Cruciate Ligament Reconstruction. Am. J. Sports Med. 44, 425-432 (2016).
34. Veras, L. et al. Mechanical loading prediction through accelerometry data during walking and running. Eur. J. Sport Sci. 0, 1-10 (2022).
35. Alcantara, R. S., Edwards, W. B., Millet, G. Y. & Grabowski, A. M. Predicting continuous ground reaction forces from accelerometers during uphill and downhill running: a recurrent neural network solution [PeerJ]. PeerJ 10, e12752.
36. Jiang, X., Napier, C., Hannigan, B., Eng, J. J. & Menon, C. Estimating Vertical Ground Reaction Force during Walking Using a Single Inertial Sensor. Sensors 20, 4345 (2020).
37. Bach, M. M., Dominici, N. & Daffertshofer, A. Predicting vertical ground reaction forces from 3D accelerometery using reservoir computers leads to accurate gait event detection. 2022.02.14.480318 Preprint at https://doi.org/I0.1101/2022.02.14.480318 (2022).
38. Evans-Pickett, A. et al. Biomechanical effects of manipulating peak vertical ground reaction force throughout gait in individuals 6-12 months after anterior cruciate ligament reconstruction. Clin. Biomech. 76, 105014 (2020).
39. Pedregosa, F. et al. Scikit-learn: Machine Learning in Python. J. Mach. Learn. Res. 12, 2825-2830 (2011).
40. Gard, S. A., Miff, S. C. & Kuo, A. D. Comparison of kinematic and kinetic methods for computing the vertical motion of the body center of mass during walking. Hum. Mov. Sci. 22, 597-610 (2004).
41. Ribeiro, M. T., Singh, S. & Guestrin, C. ‘Why Should I Trust You?’: Explaining the Predictions of Any Classifier. Preprint at https://doi.org/10.48550/arXiv.1602.04938 (2016).

What is claimed is:

1. A method for detecting and monitoring gait using a portable gait biofeedback system, the method comprising:
receiving, by a central host computing device and from a plurality of wearable sensor devices, sensor signal data representative of biometric gait data of a subject;
combining, by the central host computing device, the sensor signal data from each of the wearable sensor devices to generate a gait data collection in real-time;
providing, by the central host computing device, the gait data collection to a cloud-based biometric prediction engine; and
processing, by the biofeedback prediction engine, the gait data collection to generate vertical ground reaction force (vGRF) biofeedback prediction data associated with the subject in real-time.

2. The method of claim 1 wherein the wearable sensor devices include one wearable sensor device configured to be mounted on a waist of the subject and two wearable sensor devices configured to be mounted on either ankle of the subject.

3. The method of claim 2 wherein the wearable sensor device configured to be mounted on the waist includes an accelerometer.

4. The method of claim 1 wherein the wearable sensor devices include wearable sensor devices configured to be mounted on thighs or feet of the subject.

5. The method of claim 1 wherein the gait collection data includes at least one of motion data or bilateral pressure data.

6. The method of claim 1 wherein the biofeedback prediction engine is executed by a machine learning algorithm.

7. The method of claim 1 wherein the plurality of wearable sensor devices includes at least one of: a motion sensor device, a force measurement sensor device, an accelerometer device, an inertial measurement unit (IMU) device, or a plantar pressure sensor device.

8. The method of claim 1 wherein the vGRF biofeedback prediction data is provided to the central host computing device for visual display on a user interface in real-time.

9. The method of claim 1 wherein the vGRF biofeedback prediction data includes a visual representation of one or more threshold levels of ground reaction force that compels the subject to alter step-by-step gait biomechanics in real-time.

10. The method of claim 1 wherein the vGRF biofeedback prediction data is provided to the central host computing device for auditory notifications on a user interface in real-time.

11. A portable gait biofeedback system for detecting and monitoring gait, the system comprising:
a plurality of wearable sensor devices applied to a subject and configured to monitor for biometric gait data of the subject and generate sensor signal data representative of the biometric gait data;
a central host computing device configured for receiving, from the plurality of wearable sensor devices, the sensor signal data and combining the sensor signal data from each of the wearable sensor devices to generate a gait data collection in real-time; and
a biofeedback prediction engine configured to receive the gait data collection from the host central computing device and to process the gait data collection to generate vertical ground reaction force (vGRF) biofeedback prediction data associated with the subject in real-time.

12. The system of claim 11 wherein the wearable sensor devices include one wearable sensor device configured to be mounted on a waist of the subject and two wearable sensor devices configured to be mounted on either ankle of the subject.

13. The system of claim 12 wherein the wearable sensor device configured to be mounted on the waist includes an accelerometer.

14. The system of claim 11 wherein the wearable sensor devices include wearable sensor devices configured to be mounted on thighs or feet of the subject.

15. The system of claim 11 wherein the gait collection data includes at least one of motion data or bilateral pressure data.

16. The system of claim 11 wherein the biofeedback prediction engine is executed by a machine learning algorithm.

17. The system of claim 11 wherein the plurality of wearable sensor devices includes at least one of: a motion sensor device, a force measurement sensor device, an accelerometer device, an inertial measurement unit (IMU) device, or a plantar pressure sensor device.

18. The system of claim 11 wherein the vGRF biofeedback prediction data is provided to the central host computing device for visual display on a user interface in real-time.

19. The system of claim 11 wherein the vGRF biofeedback prediction data includes a visual representation of one or more threshold levels of ground reaction force that compels the subject to alter step-by-step gait biomechanics in real-time.

20. The system of claim 11 wherein the vGRF biofeedback prediction data is provided to the central host computing device for auditory notifications on a user interface in real-time.

21. One or more non-transitory computer readable media having stored thereon executable instructions that when executed by at least one processor of a computer cause the computer to perform steps comprising:
receiving, by a central host computing device and from a plurality of wearable sensor devices, sensor signal data representative of biometric gait data of a subject;
combining, by the central host computing device, the sensor signal data from each of the wearable sensor devices to generate a gait data collection in real-time;
providing, by the central host computing device, the gait data collection to a cloud-based biometric prediction engine, and
processing, by the biofeedback prediction engine, the gait data collection to generate vertical ground reaction force (vGRF) biofeedback prediction data associated with the subject in real-time.

22. The one or more non-transitory computer readable media of claim 21 wherein the wearable sensor devices include one wearable sensor device configured to be mounted on a waist of the subject and two wearable sensor devices configured to be mounted on either ankle of the subject.
